(19) United States
(12) Patent Application Publication
(10) Pub. No.: US 2008/0311066 A1
(43) Pub. Date: Dec. 18, 2008

(54) Cosmetics Compositions Comprising at Least One Surfactant and at Least One Novel Ethylene Copolymer with Polyethylene Glycol Grafts

(75) Inventors: Henri Samain, Bievres (FR); Laurence Paul, Saint Leu La Foret (FR); Gwenaelle Jegou, Livry Gargan (FR)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: L'ORÉAL, Paris (FR)

(21) Appl. No.: 11/631,117

(22) PCT Filed: Jul. 1, 2005
(86) PCT No.: PCT/FR05/01701

§ 371 Date: Dec. 29, 2006

Related U.S. Application Data
(60) Provisional application No. 60/586,726, filed on Jul. 12, 2004.
(60) Provisional application No. 60/587,553, filed on Jul. 14, 2004.

(30) Foreign Application Priority Data
Jul. 2, 2004 (FR) 0451409
Jul. 2, 2004 (FR) 0451411

(51) Int. Cl. A61K 8/91; A61Q 5/02; A61K 8/81

(52) U.S. Cl. 424/70.16; 424/70.11

(57) ABSTRACT

The invention relates to cosmetic compositions comprising:
A) at least one anionic surfactant and at least one nonionic surfactant, and
B) at least one ethylenic copolymer comprising, as a weight percentage relative to the total weight of the polymer:
   a) 10-60% by weight of one or more monomers of formula (I) as defined below;
   b) 40-90% by weight of at least one “essentially cationic” monomer chosen from:
(i) one or more cationic monomers of formula (IIa),
(ii) one or more amphoteric monomers of formulae (IIc) and (IId), and
(iii) a mixture of one or more cationic monomers of formula (IIa) with one or more anionic monomers chosen from maleic anhydride and/or those of formula (IIb); and/or with one or more amphoteric monomers chosen from those of formulae (IIc) and (IId),
c) and optionally 0-50% by weight of nonionic hydrophilic monomers, excluding methyl acrylate, methyl methacrylate and isopropyl acrylate if they are present in an amount of greater than or equal to 10% by weight.

Treated hair disentangles easily during rinsing, and is soft after drying. The compositions according to the invention also allow, once the head of hair is dry, particularly advantageous shaping of the hair.

The present invention relates to compositions comprising at least one surfactant and at least one ethylenic copolymer with polyethylene glycol grafts.

It is known practice to use polymers in cosmetics, and especially in haircare in leave-in products, for example to give the hair hold or styling.

In the field of “rinse-out” compositions, such as shampoos or hair conditioners, water-soluble synthetic cationic polymers are also used, which are known to give the hair a good cosmeticity; however, these polymers provide no hair shaping effect. This is likewise the case for cationic nature-derived polymers such as modified guar gums, which also provide a cosmetic nature without allowing shaping. In the field of rinse-out compositions, polymers do not provide sufficient styling associated with an acceptable cosmeticity.

The aim of the present invention is to provide cosmetic compositions comprising polymers capable of providing a real styling effect while at the same time maintaining an acceptable cosmeticity for the compositions, and especially sparingly viscous polymers that only slightly modify the viscosity of the compositions comprising them.

After considerable research, the Applicant has discovered that the use of polymers comprising, inter alia, monomers of the polyethylene glycol (meth)acrylate type as defined below can allow the preparation of rinse-out or leave-in styling compositions of adequate cosmetic properties.

Polymers containing polyethylene glycol (meth)acrylate (MPEG) units are described in the prior art.

Thus, EP 372 546 discloses copolymers based on MPEG and monomers of C1-C8 alkyl (meth)acrylamide type, which may comprise cationic monomers. However, these polymers comprise only a small proportion of cationic monomers, which does not allow them to generate adequate cosmetic effects, especially deposition on the hair that is sufficient to provide the desired properties.

Document JP2002-322 219 describes polymers containing MPEG units in combination with hydrophobic monomers based on polypropylene glycol (PPO) or polytetramethylene oxide, and cationic monomers. However, it has been found that these polymers comprising hydrophobic monomers do not allow satisfactory cosmetic properties to be obtained.

A composition comprising cationic polymers in which the monomers of PEG type are combined with monomers comprising quaternary amine units is also known from patent JP2002-284 627. However, the presence of quaternary units may induce, gradually in the course of applications, extra deposition that may, in certain cases, harm the cosmetic quality of the composition. Moreover, these polymers contain a low content of cationic charge, of about 0.5% to 6%, which does not allow optimum affinity for the hair.

Document JP2000-302 649 describes a haircare composition comprising a polymer that comprises cationic or amphoteric monomers, monomers with a polyether group, especially of PEG or PPO type, and also optional monomers that may be mainly hydrophobic (for example stearyl methacrylate).

Haircare compositions comprising a polymer that comprises monomers of MPEG type in combination with ionic, cationic or amphoteric monomers, and additional monomers of C1-C24 alkyl (meth)acrylate type, which are mainly hydrophobic, are also known from patent JP07-285 831. However, the presence of hydrophobic comonomers, for example of butyl or stearyl acrylate type, does not make it possible to obtain adequate cosmetic properties, and especially does not make it possible to obtain good disentangling of wet hair, just after shampooing.

Patent application WO 03/075 867 is also known, which describes linear block copolymers comprising a poly(alkylene glycol) block surrounded by two ethylenic blocks. These polymers have the drawback of having a central block of poly(alkylene glycol) type of high mass, which gives the polymer high crystallinity, which may lead to opaque products and/or products of greasy nature.

The Applicant has discovered novel polymers that can give a styling and conditioning effect to cosmetic haircare products.

Without wishing to be bound by the present explanation, it may be considered that this may be due especially to the presence of PEG (meth)acrylate (MPEG) units in the polymer chain, these units largely contributing to the obtained effect. Specifically, it has been found that this effect is not obtained with a simple mixture of cationic polymer and of polymer of PEG type.

Surprisingly, the polymers according to the invention have advantageous cosmetic properties, for example during application in a shampoo composition comprising a combination of particular surfactants; specifically, it has been found that the hair disentangles easily during shampooing, and is soft; after drying, the compositions according to the invention also allow, once the hair has dried, particularly advantageous shaping of the hair.

The cosmetic compositions according to the invention are characterized in that they comprise:
I) at least one anionic surfactant and at least one nonionic surfactant, and
II) at least one ethylenic copolymer comprising, as a weight percentage relative to the total weight of the polymer:
a) 10-60% by weight of one or more monomers of formula (I) as defined below;
b) 40-90% by weight of at least one “essentially cationic” monomer chosen from:
   (i) one or more cationic monomers of formula (IIa),
   (ii) one or more amphoteric monomers of formulae (IIc) and (IId), and
   (iii) a mixture of one or more cationic monomers of formula (IIa) with one or more anionic monomers chosen from maleic anhydride and/or those of formula (IIb); and/or with one or more amphoteric monomers chosen from those of formulae (IIc) and (IId),
c) and optionally 0-50% by weight of nonionic hydrophilic monomers, excluding methyl acrylate, methyl methacrylate and isopropyl acrylate if they are present in an amount of greater than or equal to 10% by weight.

In the rest of the present description, the term “cyclic radical” means a monocyclic or polycyclic radical, which may be in the form of one or more saturated and/or unsaturated, optionally substituted rings (for example cyclohexyl, cyclodecyl, benzyl or fluorenyl), but also a radical that comprises one or more of the said rings (for example p-tert-butyl-cyclohexyl or 4-hydroxybenzyl).

The term “saturated and/or unsaturated radical” means totally saturated radicals, totally unsaturated radicals, including aromatic radicals, and also radicals comprising one or more double and/or triple bonds, the rest of the bonds being single bonds.

Anionic surfactants, among which mention may be made, alone or as mixtures, of salts (in particular alkali-metal salts, especially sodium salts, ammonium salts, amine salts, amino alcohol salts or magnesium salts) of the following compounds: alkyl sulfates, alkyl ether sulfates, alkylamido ether sulfates, alkylarylpolyether sulfates, monoglyceride sulfates; alkyl sulfonates, alkyl phosphates, alkylamide sulfonates, alkylaryl sulfonates, α-olefin sulfonates, paraffin sulfonates; alkyl sulfosuccinates, alkyl ether sulfosuccinates, alkylamide sulfosuccinates; alkyl sulfosuccinamates; alkyl sulfoacetates; alkyl ether phosphates; acyl sarcosinates; acyl isethionates and N-acyltaurates, the alkyl or acyl radical of all of these various compounds preferably containing from 8 to 24 carbon atoms, and the aryl radical preferably denoting a phenyl or benzyl group. Mention may also be made of fatty acid salts such as the salts of oleic, ricinoleic, palmitic and stearic acids, coconut oil acid or hydrogenated coconut oil acid; acyl lactylates in which the acyl radical contains 8 to 20 carbon atoms; alkyl-D-galactosiduronic acids and their salts, as well as polyoxyalkylenated (C₆-C₂₄) alkyl ether carboxylic acids, polyoxyalkylenated (C₆-C₂₄) alkylaryl ether carboxylic acids, polyoxyalkylenated (C₆-C₂₄) alkylamido ether carboxylic acids and their salts, in particular those containing from 2 to 50 ethylene oxide groups, and mixtures thereof. Fatty acids are not preferred.

Among the anionic surfactants, it is preferred to use alkyl sulfate salts, alkyl ether sulfates and alkyl ether carboxylates, and mixtures thereof, in particular in the form of salts of alkali metals (N or K) or alkaline-earth metals (e.g. Mg), or of ammonium, of amine or of amino alcohol, and mixtures thereof.

The amount of anionic surfactants will preferably be from 3% to 40% by weight and in particular from 5% to 25% by weight relative to the total weight of the cosmetic composition.

Nonionic surfactants, among which mention may be made, alone or as mixtures, of polyethoxylated, polypropoxylated or polyglycerolated fatty acids, alkylphenols, α-diols or alcohols having a fatty chain containing, for example, 8 to 22 carbon atoms, it being possible for the number of ethylene oxide or propylene oxide groups to range in particular from 2 to 50 and for the number of glycerol groups to range in particular from 2 to 30. Mention may also be made of copolymers of ethylene oxide and of propylene oxide, condensates of ethylene oxide and of propylene oxide with fatty alcohols; polyethoxylated fatty amides preferably having from 2 to 30 mol of ethylene oxide, polyglycerolated fatty amides containing on average 1 to 5, and in particular 1.5 to 4, glycerol groups; oxyethylenated fatty acid esters of sorbitan having from 2 to 30 mol of ethylene oxide; fatty acid esters of sucrose, fatty acid esters of polyethylene glycol, alkylpolyglycosides, N-alkylglucamine derivatives, amine oxides such as (C₁₀-C₁₄)alkylamine oxides or N-acylaminopropylmorpholine oxides.

Preferably, the nonionic surfactant is chosen from:
glycerolated fatty alcohols;
alkylpolyglycosides.

The term “fatty chain” means a linear or branched, saturated or unsaturated hydrocarbon-based chain containing from 6 to 30 and preferably from 8 to 24 carbon atoms.

As regards the alkylpolyglycosides, these compounds are well known and may be represented more particularly by the following general formula:

R₁O—(R₂O)ₜ(G)ᵥ (II)

in which R₁ represents a linear or branched alkyl and/or alkenyl radical containing from about 8 to 24 carbon atoms, an alkylphenyl radical in which the linear or branched alkyl radical contains from 8 to 24 carbon atoms, R₂ represents an alkylene radical containing from about 2 to 4 carbon atoms, G represents a sugar unit containing 5 or 6 carbon atoms, t denotes a value ranging from 0 to 10 and preferably from 0 to 4, and v denotes a value ranging from 1 to 15.

Alkylpolyglycosides that are preferred according to the present invention are compounds of formula (II) in which R₁ more particularly denotes a saturated or unsaturated, linear or branched alkyl radical containing from 8 to 18 carbon atoms, t denotes a value ranging from 0 to 3 and even more particularly equal to 0, and G may denote glucose, fructose or galactose, preferably glucose. The degree of polymerization, i.e. the value of v in formula (II), may range from 1 to 15 and preferably from 1 to 4. The mean degree of polymerization is more particularly between 1 and 2 and even more preferentially from 1.1 to 1.5. The glycoside bonds between the sugar units are of 1-6 or 1-4 type and preferably of 1-4 type.

Compounds of formula (II) are especially represented by the products sold by the company Cognis under the names Plantaren® (600 CS/U, 1200 and 2000) or Plantacare® (818, 1200 and 2000). It is also possible to use the products sold by the company SEPPIC under the names Triton CG 110 (or Oramix CG 110) and Triton CG 312 (or Oramix® NS10), the products sold by the company BASF under the name Lutensol GD 70 or the products sold by the company Chem Y under the name AG10 LK.

It is also possible to use, for example, C8/C16 alkyl polyglucoside-1,4 as an aqueous 53% solution sold by Cognis under the reference Plantacare® 818 UP.

As regards the monoglycerolated or polyglycerolated surfactants, they preferably comprise on average from 1 to 30 glycerol groups, more particularly from 1 to 10 and in particular from 1.5 to 5 glycerol groups.

The monoglycerolated or polyglycerolated surfactants are preferably chosen from the following compounds of formulae: RO[CH₂CH(CH₂OH)O]ₘH, RO[CH₂CH(OH)CH₂O]ₘH or RO[CH(CH₂OH)CH₂O]ₘH; in which R represents a saturated or unsaturated, linear or branched hydrocarbon-based radical containing from 8 to 40 carbon atoms and preferably from 10 to 30 carbon atoms; m is an integer between 1 and 30, preferably between 1 and 10 and more particularly from 1.5 to 6.

R may optionally comprise hetero atoms, for instance oxygen and nitrogen. In particular, R may optionally comprise one or more hydroxyl and/or ether and/or amide groups.

R preferably denotes optionally monohydroxylated or polyhydroxylated C₁₀-C₂₀ alkyl and/or alkenyl radicals.

The polyglycerolated (3.5 mol) hydroxylauryl ether sold under the name Chimexane® NF from Chimex may be used, for example.

Among the nonionic surfactants, C₆-C₂₄ alkyl polyglucosides and more particularly C₈-C₁₆ alkyl polyglucosides are preferably used.

The total amount of nonionic surfactants preferably ranges from 0.5% to 25% by weight, in particular from 1% to 20% by weight and more particularly from 2% to 10% by weight relative to the total weight of the cosmetic composition.

Preferably, the composition may also comprise at least one amphoteric surfactant.

Amphoteric surfactants, among which mention may be made, alone or as mixtures, of aliphatic secondary or tertiary amine derivatives in which the aliphatic radical is a linear or branched chain containing 8 to 22 carbon atoms and containing at least one water-soluble anionic group (for example carboxylate, sulfonate, sulfate, phosphate or phosphonate); mention may also be made of (C₈-C₂₀)alkylbetaines, sulfo-betaines, (C₈-C₂₀)alkylamido(C₁-C₆)alkylbetaines such as cocoamidopropylbetaine, or (C₈-C₂₀) alkylamido (C₁-C₆)-alkylsulfobetaines.

Among the amphoteric surfactants, C₈-C₂₀ alkyl betaines, (C₈-C₂₀)alkylamido(C₆-C₈)alkyl betaines and alkylamphodiacetates, and mixtures thereof, are preferably used.

The total amount of amphoteric surfactants preferably ranges from 0.5% to 20% by weight, in particular from 1% to 10% by weight and more particularly from 1% to 5% by weight relative to the total weight of the cosmetic composition.

Among the mixtures of surfactants that may be used, combinations of anionic surfactants, nonionic surfactants and amphoteric surfactants are preferred.

The anionic surfactant preferably used is chosen from sodium, triethanolamine or ammonium (C₁₂-C₁₄)alkyl sulfates, sodium, triethanolamine or ammonium (C₁₂-C₁₄)alkyl ether sulfates oxyethylenated with 2.2 mol of ethylene oxide, sodium cocoyl isethionate and sodium α-(C₁₄-C₁₆)olefin sulfonate, and mixtures thereof with a nonionic surfactant and:
either an amphoteric surfactant such as the amine derivatives known as disodium cocoamphodipropionate or sodium cocoamphopropionate sold especially by the company Rhodia Chimie under the trade name Miranol C2M CONC as an aqueous solution containing 38% active material, or under the name Miranol C32;
or an amphoteric surfactant of zwitterionic type such as alkylbetaines, in particular the cocobetaine sold under the name Dehyton AB 30 as an aqueous solution containing 32% AM by the company Cognis.

The total amount of surfactants especially ranges from 3.5% to 50% by weight, preferably from 5% to 30% by weight and even more preferentially from 8% to 25% by weight relative to the total weight of the composition.

### Ethylenic Copolymer:

The ethylenic copolymer according to the invention thus comprises at least one monomer of formula (I), which may be present alone or as a mixture:

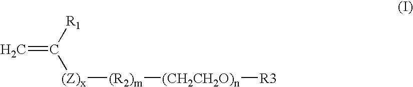

in which:
R1 is a hydrogen atom or a linear or branched hydrocarbon-based radical, of the type CₚH₂ₚ₊₁, with p being an integer between 1 and 12 inclusive;
Z is a divalent group chosen from —COO—, —CONH—, —CONCH₃—, —OCO—, —O—, —SO₂—, —CO—C—CO— and —CO—CH₂—CO—;
x is 0 or 1;
R2 is a saturated or unsaturated, optionally aromatic, linear, branched or cyclic carbon-based divalent radical of 1 to 30 carbon atoms, possibly comprising 1 to 18 heteroatoms chosen from O, N, S, F, Si and P;
m is 0 or 1;
n is an integer between 3 and 300 inclusive;
R3 is a hydrogen atom or a saturated or unsaturated, optionally aromatic, linear, branched or cyclic carbon-based radical of 1 to 30 carbon atoms, possibly comprising 1 to 20 heteroatoms chosen from O, N, S, F, Si and P;
   and salts thereof.

R1 may especially represent a methyl, ethyl, propyl or butyl radical. Preferably R1 represents hydrogen or a methyl radical.

Preferably, Z represents COO or CONH.

Preferably, x is equal to 1. In the radical R2, the heteroatom(s), when they are present, may be intercalated in the chain of said radical R2, or alternatively said radical R2 may be substituted with one or more groups comprising them such as hydroxy or amino (NH2, NHR′ or NR′R″ with R′ and R″, which may be identical or different, representing a linear or branched C1-C22 alkyl, especially methyl or ethyl).

R2 may especially be:
an alkylene radical such as methylene, ethylene, propylene, n-butylene, isobutylene, tert-butylene, n-hexylene, n-octylene, n-dodecylene, n-octadecylene, n-tetradecylene or n-docosanylene;
a phenylene radical —C₆H₄— (ortho, meta or para), optionally substituted with a C1-C12 alkyl radical optionally comprising 1 to 25 heteroatoms chosen from O, N, S, F, Si and P; or alternatively a benzylene radical —C₆H₄—CH₂—, optionally substituted with a C1-C12 alkyl radical optionally comprising 1 to 8 heteroatoms chosen from O, N, S, F, Si and P;
a pyridinium radical of formula:

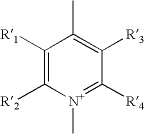

with R′¹ to R′⁴, which may be identical or different, chosen from H and a C1-C12 alkyl radical optionally comprising 1 to 8 heteroatoms chosen from O, N, S, F, Si and P; R′¹ to R′⁴ may especially be methyl and/or ethyl;
a radical of formula —CH₂—O—CO—O—, CH₂—CH₂—O—CO—O—, —CH₂—CO—O—, —CH₂—CH₂—CO—O—, —CH₂—O—CO—NH—, —CH₂—CH₂—O—CO—NH—; —CH₂—NH—CO—NH—, —CH₂—CH₂—NH—CO—NH—; —CH₂—CHOH—, —CH₂—CH₂—CHOH—, —CH₂—CH₂—CH(NH₂)—, —CH₂—CH(NH₂)—, —CH₂—CH₂—CH(NHR′)—, —CH₂—CH(NHR′)—, —CH₂—CH₂—CH(NR′R″)-, —CH₂—CH(NR′R″)-, —CH₂—CH₂—CH₂—NR′—, —CH₂—CH₂—CH₂—O—; —CH₂—CH₂—CHR′—O— with R′ and R″ representing a linear or branched C1-C22 alkyl optionally comprising 1 to 12 heteroatoms chosen from O, N, S, F, Si and P;
or a mixture of these radicals.

Preferably, n is between 5 and 200 inclusive and better still between 7 and 100 inclusive, or even between 9 and 50 inclusive.

Preferably, R3 is a hydrogen atom; a benzyl or phenyl radical optionally substituted with a C1-C12 alkyl radical optionally comprising 1 to 8 heteroatoms chosen from O, N, S, F, Si and P; a C1-C30 and especially C1-C22 or even C2-C16 alkyl radical, optionally comprising 1 to 18 heteroatoms chosen from O, N, S, F, Si and P.

These benzyl, phenyl or alkyl radicals may especially comprise a function chosen from the following functions:

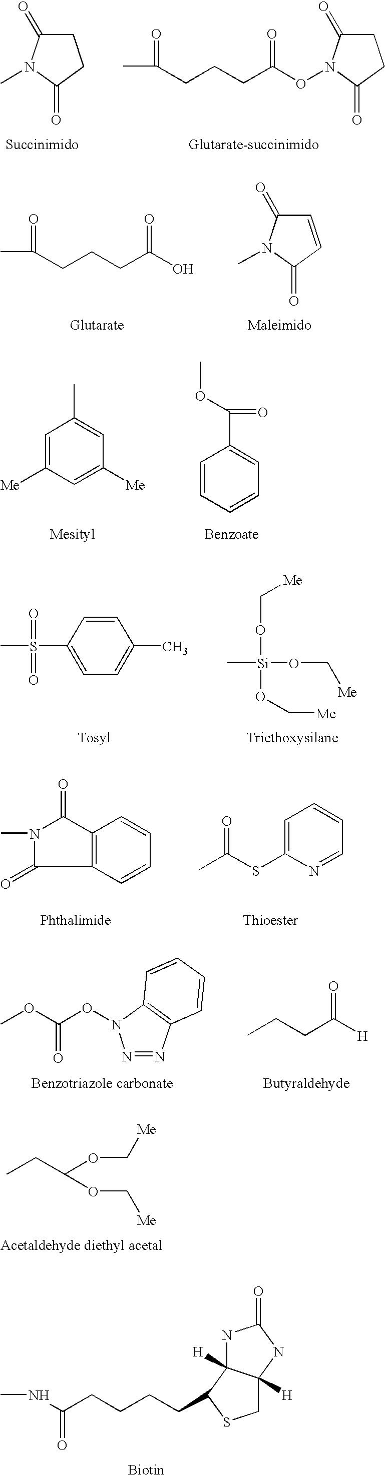

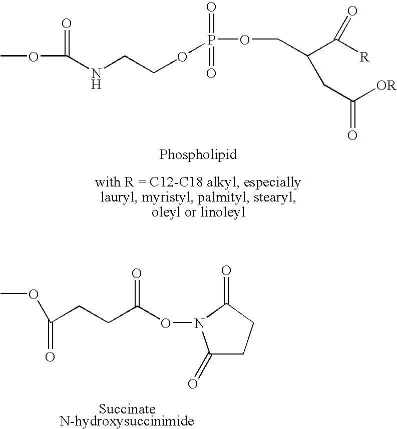

or alternatively chosen from —SO₃H, —COOH, —PO₄, —NR5R6 and —N⁺R5R6R7, with R5, R6 and R7, independently of each other, chosen from H and linear, branched or cyclic C1-C18 alkyls, especially methyl optionally comprising one or more heteroatoms or alternatively bearing protective groups such as t-butyloxycarbonyl (also known as BOC) or 9-fluorenylmethoxycarbonyl (also known as FmoC).

Among the radicals R3, mention may be made of methyl, ethyl, propyl, benzyl, ethylhexyl, lauryl, stearyl and behenyl (—(CH₂)₂₁—CH₃) chains, and also fluoroalkyl chains, for instance heptadecafluorooctylsulfonyl-aminoethyl CF₃— (CF₂)₇—SO₂—N(C₂H₅)—CH₂—CH₂; or alternatively —CH₂—CH₂—CN, succinimido, maleimido, mesityle, tosyl, triethoxysilane or phthalimide chains.

The amine units and/or the anionic groups of the monomer of formula (I) may optionally be neutralized.

The amine units of the monomer may optionally be neutralized.

Among the salts, mention may be made of the salts of mineral acids, such as sulfuric acid, hydrochloric acid, hydrobromic acid, hydroiodic acid, phosphoric acid or boric acid. Mention may also be made of the salts of organic acids, which may comprise one or more carboxylic, sulfonic or phosphonic acid groups. These may be linear, branched or cyclic aliphatic acids or alternatively aromatic acids. These acids may also comprise one or more heteroatoms chosen from O and N, for example in the form of hydroxyl groups. Mention may be made especially of propionic acid, acetic acid, terephthalic acid, citric acid and tartaric acid.

Neutralization of the anionic groups may be performed with a mineral base such as LiOH, NaOH, KOH, Ca(OH)₂, NH₄OH, Mg(OH)₂ or Zn(OH)₂; or with an organic base such as a primary, secondary or tertiary alkylamine, especially triethylamine or butylamine. This primary, secondary or tertiary alkylamine may comprise one or more nitrogen and/or oxygen atoms and may thus comprise, for example, one or more alcohol functions; mention may be made especially of 2-amino-2-methyl-propanol, triethanolamine and dimethylamino-2-propanol. Mention may also be made of lysine or 3-(dimethylamino)propylamine.

Among the monomers of formula (I) that are particularly preferred, mention may be made of:
poly(ethylene glycol) (meth)acrylate in which R1 is H or methyl; Z is COO, x=1, m=0 and R3=H;
methylpoly(ethylene glycol) (meth)acrylate, also known as methoxypoly(ethylene glycol) (meth)acrylate, in which R1 is H or methyl, Z is COO, x=1, m=0 and R3=methyl;
alkylpoly(ethylene glycol) (meth)acrylates in which R1 is H or methyl, Z is COO, x=1, m=0 and R3=alkyl;
phenylpoly(ethylene glycol) (meth)acrylate, also known as poly(ethylene glycol) phenyl ether (meth)acrylate, in which R1 is H or methyl, Z is COO, x=1, m=0 and R3=phenyl;
the following monomer:

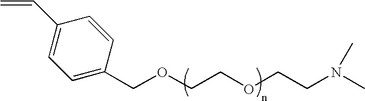

in which n is preferably between 3 and 100 inclusive and especially 5 to 50 inclusive, or even 7 to 30 inclusive.

The monomers of formula (I) that are most particularly preferred are chosen from poly(ethylene glycol) (meth)acrylates and methylpoly(ethylene glycol) (meth)acrylates, preferably those with a molecular weight of between 350 and 15 000 g/mol and especially between 500 and 8000 g/mol.

Poly(ethylene glycol) (meth)acrylates are most particularly preferred, and in particular those with a molecular weight of between 350 and 15 000 g/mol and especially between 500 and 8000 g/mol.

Examples of Commercial Monomers are:
CD 350 (methoxypoly(ethylene glycol 350) methacrylate) and CD 550 (methoxypoly(ethylene glycol 550) methacrylate), sold by Sartomer Chemicals;
M90G (methoxypolyethylene glycol methacrylate (9 repeater units)) and M230G (methoxypolyethylene glycol methacrylate (23 repeating units)) available from Shin-Nakamura Chemicals;
methoxypoly(ethylene glycol) methacrylates with average molecular weights of 300, 475 or 1100, available from Sigma-Aldrich;
methoxypoly(ethylene glycol)acrylate with an average molecular weight of 426, available from Sigma-Aldrich;
methoxypoly(ethylene glycol) methacrylates available from Laporte under the trade names: MPEG 350, MPEG 550, S10W, S20W;
poly(ethylene glycol) monomethylether, mono(succinimidyl succinate) ester with an average molecular weight of 1900 or 5000, from Polysciences;
behenylpoly(ethylene glycol PEG-25) methacrylate, available from Rhodia under the name Sipomer BEM;
poly(ethylene glycol)phenylether acrylates with average molecular weights of 236, 280 or 324, available from Aldrich;
methoxypolyethylene glycol 5000 2-(vinylsulfonyl)ethyl ether available commercially from Fluka;
polyethylene glycol ethyl ether methacrylate available from Aldrich;
polyethylene glycol 8000, 4000, 2000 methacrylates from Monomer & Polymer Dajac Laboratories;
polyethylene glycol N-hydroxysuccinimide vinyl sulfone available commercially from Nektar Molecule Engineering (Shearwater).

Preferably, the monomer of formula (I) has a molecular weight of between 350 and 15 000 g/mol and especially between 500 and 8000 g/mol.

The monomer of formula (I), alone or as a mixture, is present in a proportion of from 10% inclusive to 60% exclusive by weight, especially from 20% inclusive to 55% inclusive by weight and preferably from 30% inclusive to 50% inclusive by weight, relative to the weight of the final polymer.

### Ionic Units

The ethylenic copolymer according to the invention also comprises at least one “essentially cationic” monomer, or a salt thereof, chosen from:
(i) one or more cationic monomers of formula (IIa),
(ii) one or more amphoteric monomers of formulae (IIc) and (IId), and
(iii) a mixture of one or more cationic monomers of formula (IIa) with one or more anionic monomers chosen from maleic anhydride and/or those of formula (IIb); and/or with one or more amphoteric monomers chosen from those of formulae (IIc) and (IId).

Preferably, the “essentially cationic” monomer is chosen from the cationic monomers of formula (IIa) and the amphoteric monomers of formula (IIc) or (IId) and preferentially from the cationic monomers of formula (IIa).

The term “cationic monomer” means a monomer comprising units capable of bearing a cationic charge in the pH range of between 3 and 12. These units do not necessarily have a permanent charge irrespective of the pH. The cationic unit does not need to be protonated at each of these pH values.

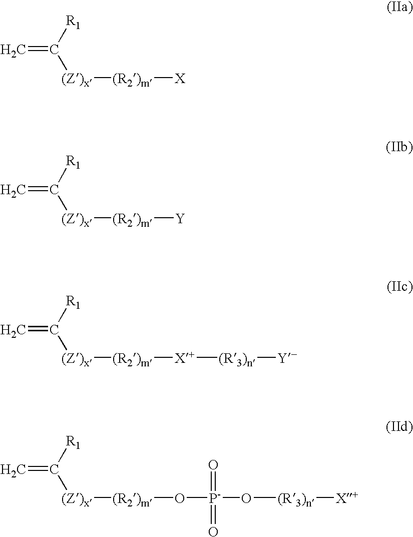

in which:
R1 is a hydrogen atom or a linear or branched hydrocarbon-based radical of the type CₚH₂ₚ₊₁, with p being an integer between 1 and 12 inclusive.

R1 may especially represent a methyl, ethyl, propyl, isopropyl, n-butyl, isobutyl or tert-butyl radical. Preferably, R1 represents hydrogen or a methyl radical.
Z′ is a divalent group chosen from —COO—, —CONH—, —CONCH₃—, —OCO— or —O—, —SO₂—-CO—O—CO— or —CO—CH₂—CO—.

Preferably, Z′ is chosen from COO and CONH.
x′ is 0 or 1, preferably 1.
R′2 is a saturated or unsaturated, optionally aromatic, linear, branched or cyclic divalent carbon-based radical of 1 to 30 carbon atoms, possibly comprising 1 to 18 heteroatoms chosen from O, N, S, F, Si and P.

In the radical R′2, the heteroatom(s), when they are present, may be intercalated in the chain of said radical R′2, or alternatively said radical R′2 may be substituted with one or more groups comprising them such as hydroxyl or amino (NH2, NHR′ or NR′R″ with R′ and R″, which may be identical or different, representing a linear or branched C1-C22 alkyl, especially methyl or ethyl).

R′2 may especially be:
an alkylene radical such as methylene, ethylene, propylene, n-butylene, isobutylene, tert-butylene, n-hexylene, n-octylene, n-dodecylene, n-octadecylene, n-tetradecylene or n-docosanylene;
a phenylene radical —C₆H₄— (ortho, meta or para), optionally substituted with a C1-C12 alkyl radical optionally comprising 1 to 25 heteroatoms chosen from N, O, S, F, Si and/or P; or alternatively a benzylene radical —C₆H₄—CH₂—, optionally substituted with a C1-C12 alkyl radical optionally comprising 1 to 25 heteroatoms chosen from O, N, S, F, Si and P;
a radical of formula —CH₂—O—CO—O—, CH₂—CH₂—O—CO—O—, —CH₂—CO—O—, —CH₂—CH₂—CO—O—, —[(CH₂)₅—CO—O]ₙ—, —CH₂—CH(CH₃)—O—, —(CH₂)₂—O—, —CH₂—O—CO—NH—, —CH₂—CH₂—O—CO—NH—; —CH₂—NH—CO—NH— or —CH₂—CH₂—NH—CO—NH—, —CH₂—CHOH—, —CH₂—CH₂—CHOH—, —CH₂—CH₂—CH(NH₂)—, —CH₂—CH(NH₂)—, —CH₂—CH₂—CH(NHR′)—, —CH₂—CH(NHR′)— —CH₂—CH₂—CH(NR′R″)-, —CH₂—CH(NR′R″)-, —CH₂—CH₂—CH₂—NR′—, —CH₂—CH₂—CH₂—O—; —CH₂—CH₂—CHR′—O— with R′ and R″ representing a linear or branched C1-C22 alkyl optionally comprising 1 to 12 heteroatoms chosen from O, N, S, F, Si and P;
or a mixture of these radicals;
m′ is 0 or 1;
X (in formula IIa) is a group of formula —N(R₆)(R₇) or —P(R₆)(R₇) or —P+R₆R₇R₈, with R₆, R₇ and R₈ representing, independently of each other, either (i) a hydrogen atom, or (ii) a linear, branched or cyclic, saturated or unsaturated, optionally aromatic alkyl group containing from 1 to 18 carbon atoms, possibly comprising 1 to 10 heteroatoms chosen from O, N, S, F, Si and P; or (iii) R6 and R7 may form with the nitrogen or phosphorus atom a first saturated or unsaturated, optionally aromatic ring comprising in total 5, 6, 7 or 8 atoms, and especially 4, 5 or 6 carbon atoms and/or 2 to 4 heteroatoms chosen from O, S and N; said first ring possibly being fused with one or more other saturated or unsaturated, optionally aromatic rings each comprising 5, 6 or 7 atoms, and especially 4, 5, 6 or 7 carbon atoms and/or 2 to 4 heteroatoms chosen from O, S and N.

For example, R6 and R7 may be chosen from hydrogen and a methyl, ethyl, propyl, isopropyl, n-butyl, t-butyl, isobutyl, octyl, lauryl or stearyl group.

Preferably, R6 and R7 are chosen, independently of each other, from H, CH3 and C2H5.

Alternatively, X may represent a group —R′6-N—R′7- in which R′6 and R′7 form with the nitrogen atom a saturated or unsaturated, optionally aromatic ring comprising in total 5, 6, 7 or 8 atoms, and especially 4, 5 or 6 carbon atoms and/or 2 to 4 heteroatoms chosen from O, S and N; said ring possibly being fused with one or more other saturated or unsaturated, optionally aromatic rings, each comprising 5, 6 or 7 atoms, and especially 4, 5, 6, 7 or 8 carbon atoms and/or 2 to 4 heteroatoms chosen from O, S and N.

For example, X may constitute an aromatic or nonaromatic ring comprising a tertiary amine group or may represent an aromatic or nonaromatic heterocycle containing a tertiary nitrogen.

Among these preferred radicals X, mention may be made of radicals of pyridine, indolyl, isoindolinyl, imidazolyl, imidazolinyl, piperidyl, pyrazolinyl, pyrazolyl, quinoline, pyrazolinyl, pyridyl, piperazinyl, pyrrolidinyl, quinidinyl, thiazolinyl, morpholine, guanidino, amidino or phosphonium type, and mixtures thereof.

The guanidino and amidino groups are, respectively, of formula:

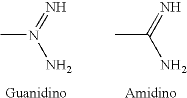

The monomers of formula (IIa) may be neutralized with neutralizers of different chemical nature.

The neutralizer may be chosen from mineral or organic acids in the Brönsted sense and preferably from organic acids. Advantageously, it may be chosen from neutralizers with a log P value of less than or equal to 2, for example between −8 and 2, preferably between −6 and 1 and especially between −6 and 0.

It may also be chosen from agents with a log P value of greater than 2, preferably greater than or equal to 2.5, especially greater than 3, and in particular between 3 and 15, or even between 3.5 and 10.

As stated hereinbelow, the log P values are known and are determined according to a standard test that determines the concentration of the neutralizer in 1-octanol and water.

The mineral acids that may be used are especially sulfuric acid, hydrochloric acid, hydrobromic acid, hydroiodic acid, phosphoric acid and boric acid.

The neutralizers of organic acid type may be chosen from linear, branched or cyclic aliphatic acids and/or unsaturated or aromatic acids, and may especially contain 1 to 1000 carbon atoms and especially 2 to 500 carbon atoms. They contain at least one acid function in the Brönsted sense, and especially one or more carboxylic, sulfonic and/or phosphonic acid groups. They may also comprise one or more heteroatoms chosen from O, N, Si, F and P, for example in the form of hydroxyl groups.

Neutralizers that may be used in particular include linear, branched or cyclic, saturated or unsaturated, optionally aromatic fatty acids containing 6 to 32 and especially 8 to 28 carbon atoms, and comprising at least one COOH or sulfonic acid (—SO₃H) function.

Linear, branched or cyclic, saturated or unsaturated, optionally aromatic hydroxy acids, especially α-hydroxy acids, containing 6 to 32 and especially 8 to 28 carbon atoms, and comprising at least one COOH or sulfonic acid (—SO₃H) function may also be used.

Alkylbenzenesulfonic acids in which the alkyl group may contain from 4 to 30 and especially from 6 to 24 carbon atoms may also be used.

Amphoteric neutralizers, especially of the alkylbetaine or alkylamidopropylbetaine type, in which the alkyl group may contain 4 to 30 and especially 6 to 24 carbon atoms, may also be used; mention may be made in particular of cocoamidopropylbetaine.

Mention may be made especially of α-hydroxyethanoic acid, α-hydroxyoctanoic acid, α-hydroxycaprylic acid, ascorbic acid, acetic acid, benzoic acid, behenic acid, capric acid, citric acid, caproic acid, caprylic acid, dodecylbenzenesulfonic acid, 2-ethylcaproic acid, folic acid, fumaric acid, galactaric acid, gluconic acid, glycolic acid, 2-hexadecyleicosanoic acid, hydroxycaproic acid, 12-hydroxystearic acid, isolauric acid (or 2-butyloctanoic acid), isomyristic acid (or 2-hexyloctanoic acid), isoarachidic acid (or 2-octyldodecanoic acid), isolignoceric acid (or 2-decyltetradecanoic acid), lactic acid, lauric acid, malic acid, myristic acid, oleic acid, palmitic acid, propionic acid, sebacic acid, stearic acid, tartaric acid, terephthalic acid, trimesic acid, undecylenic acid, propylbetaine, cocoamidopropylbetaine, and betaine hydrochloride of formula [(CH₃)₃N+CH₂CO₂H.Cl—], and mixtures thereof.

Caproic acid, 2-ethylcaproic acid, oleic acid, behenic acid, stearic acid, acetic acid, citric acid, tartaric acid, betaine hydrochloride and/or gluconic acid is preferably used as neutralizer, and preferentially betaine hydrochloride and/or behenic acid.

The log P value of certain common acids is given below for information purposes:

| | |
|---|---|
| Sulfuric acid | −1.031 +/− 0.613 |
| Acetic acid | −0.285 +/− 0.184 |
| Propionic acid | 0.246 +/− 0.184 |
| Citric acid | −1.721 +/− 0.396 |
| Gluconic acid | −3.175 +/− 0.852 |
| Boric acid | −0.292 +/− 0.753 |
| Phosphoric acid | −2.148 +/− 0.587 |
| Benzoic acid | 1.895 +/− 0.206 |
| Stearic acid | 8.216 +/− 0.186 |
| Behenic acid | 10.342 +/− 0.186 |
| Oleic acid | 7.698 +/− 0.199 |

The term “neutralization” means the action of an organic acid according to the invention, and comprising at least one acid function in the Brönsted sense, on all or part of the monomers and/or polymer mentioned above, comprising at least one basic function in the Brönsted sense.

The neutralizer, alone or as a mixture, may be added in an amount of from 0.01 to 3 and especially 0.05 to 2.5 molar equivalents, or even 0.1 to 2 molar equivalents, relative to the total amine functions of the polymer or of the monomers.

It is thus possible to underneutralize the polymer, i.e. the neutralizer may be present in an amount necessary to neutralize 1% to 99%, especially 5% to 90% or even 10% to 80% of the total amine functions of the polymer or of the monomers; this means that it is present in an amount of from 0.01 to 0.99 and especially 0.05 to 0.9 molar equivalent, or even 0.1 to 0.8 molar equivalent.

It is also possible to overneutralize the polymer, i.e. the neutralizer may be present in an amount necessary to neutralize 101% to 300%, especially from 120% to 250% or even from 150% to 200% of the total amine functions of the polymer or of the monomers; this may be the case when it is desired to ensure that the polymer has a suitable pH range and/or ionic strength with respect to the envisioned formulations. It may thus be present in an amount of from 1.01 to 3 and especially 1.2 to 2.5 molar equivalents, or even 1.5 to 2 molar equivalents, relative to the total amine functions of the polymer or of the monomers.

Preferably, the neutralizer, alone or as a mixture, is present in a stoichiometric amount relative to the total amine functions of the polymer or of the monomers; it is thus present in an amount necessary to neutralize 100% of the amine units of the polymer or of the monomers, i.e. 1 molar equivalent.

Preferentially, the nature and amount of neutralizer may be determined by a person skilled in the art so as finally to obtain a water-soluble or water-dispersible polymer.

Among the preferred monomers of formula (IIa) that may be mentioned are:

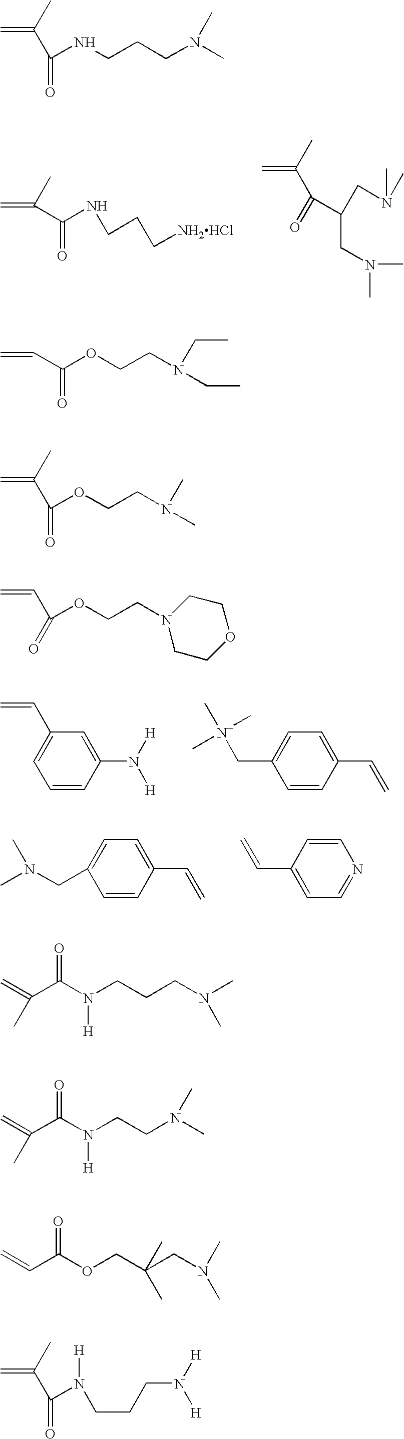

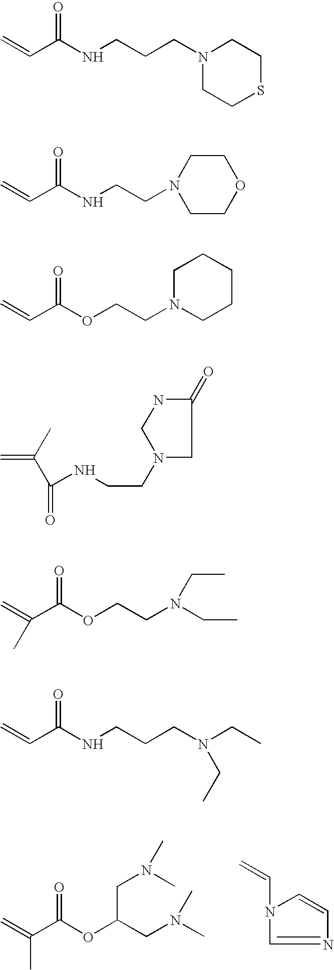

Among the monomers of formula (IIa) that are particularly preferred, mention may be made of dimethylaminopropyl(meth)acrylamide, dimethylamino-ethyl(meth)acrylamide, diethylaminoethyl (meth)acrylate, dimethylaminoethyl (meth)acrylate, vinylimidazol, vinylpyridine and morpholinoethyl (meth)acrylate, and more particularly dimethyl-aminopropyl(meth)acrylamide.

In formula (IIb), the meaning of the radicals R1, Z′, x′, R′2 and m′ is the same as that given above for formula (IIa).

In formula (IIb), Y is a group chosen from —COOH, —SO₃H, —OSO₃H, —PO₃H₂ and —OPO₃H₂.

Neutralization of the anionic groups may be performed with a mineral base, such as LiOH, NaOH, KOH, Ca(OH)₂, NH₄OH, Mg(OH)₂ or Zn(OH)₂; or with an organic base such as a primary, secondary or tertiary alkylamine, especially triethylamine or butylamine. This primary, secondary or tertiary alkylamine may comprise one or more nitrogen and/or oxygen atoms and may thus comprise, for example, one or more alcohol functions; mention may be made especially of 2-amino-2-methylpropanol, triethanolamine and dimethylamino-2-propanol. Mention may also be made of lysine or 3-(dimethylamino)propylamine.

It is understood that, according to the prior art, the SO₄H₂ and PO₄H₂ groups are linked to R′2 via the oxygen atom, whereas the SO₃H and PO₃H groups are linked to R′2 via, respectively, the S and P atoms.

Among the anionic monomers that are preferred, mention may be made of maleic anhydride and the following preferred monomers of formula (IIb): acrylic acid, methacrylic acid, crotonic acid, itaconic acid, fumaric acid, maleic acid, 2-carboxyethyl acrylate (CH2=CH—C(O)—O— (CH₂)₂—COOH); styrenesulfonic acid, 2-acrylamido-2-methylpropanesulfonic acid, vinylbenzoic acid, vinylphosphoric acid, sulfopropyl (meth)acrylate, and the salts thereof.

In formula (IIc), the meaning of the radicals R1, Z′, x′, R′2 and m′ is the same as that given above for formula (IIa).

The other radicals have the following meaning:
X′⁺ is a divalent group of formula —N⁺(R₆)(R₇)— with R6 and R7 representing, independently of each other, either (i) a hydrogen atom, or (ii) a linear, branched or cyclic, optionally aromatic alkyl group containing from 1 to 25 carbon atoms, possibly comprising 1 to 20 heteroatoms chosen from O, N, S and P; or (iii) R6 and R7 may form with the nitrogen atom a first saturated or unsaturated, optionally aromatic ring comprising in total 5, 6, 7 or 8 atoms, and especially 4, 5, 6 or 7 carbon atoms and/or 2 to 3 heteroatoms chosen from O, S and N; said first ring possibly being fused with one or more other saturated or unsaturated, optionally aromatic rings, each comprising 5, 6, 7 or 8 atoms, and especially 4, 5, 6 or 7 carbon atoms and/or 2 to 3 heteroatoms chosen from O, S and N.

For example, R6 and R7 may be chosen from hydrogen and a methyl, ethyl, propyl, isopropyl, n-butyl, t-butyl or isobutyl group.

Among the preferred radicals X′⁺ that may be mentioned are radicals of pyridine, indolyl, isoindolinyl, imidazolyl, imidazolinyl, piperidyl, pyrazolinyl, pyrazolyl, quinoline, pyrazolinyl, pyridyl, piperazinyl, pyrrolidinyl, quinidinyl, thiazolinyl, morpholine, guanidino or amidino type, and mixtures thereof.
Y′⁺ is a group chosen from —COO⁻, —SO₃⁻, —OSO₃⁻, PO₃²⁻ and OPO₃²⁻.
R′3 is a saturated or unsaturated, optionally aromatic, linear, branched or cyclic divalent carbon-based radical of 1 to 30 carbon atoms, possibly comprising 1 to 18 heteroatoms chosen from O, N, S, F, Si and P.
In the radical R′3, the heteroatom(s), when they are present, may be intercalated in the chain of said radical R′3, or alternatively said radical R′3 may be substituted with one or more groups comprising them such as hydroxyl or amino (NH₂, NHR′ or NR′R″ with R′ and R″, which may be identical or different, representing a linear or branched C1-C18 alkyl, especially methyl or ethyl).

R′3 may especially be:
an alkylene radical such as methylene, ethylene, propylene, n-butylene, isobutylene, tert-butylene, n-hexylene, n-octylene, n-dodecylene, n-octadecylene, n-tetradecylene or n-docosanylene;
a phenylene radical —C₆H₄— (ortho, meta or para), optionally substituted with a C1-C12 alkyl radical optionally comprising 1 to 5 heteroatoms chosen from O, N, S, F, Si and P; or alternatively a benzylene radical —C₆H₄—CH₂—, optionally substituted with a C1-C12 alkyl radical optionally comprising 1 to 5 heteroatoms chosen from O, N, S, F, Si and P;
a radical of formula —CH₂—O—CO—O—, CH₂—CH₂—O—CO—O—, —CH₂—CO—O—, —CH₂—CH₂—CO—O—, —[(CH₂)₅—CO—O]ₙ—, —CH₂—CH(CH₃)—O—, —(CH₂)₂—O—, —CH₂—O—CO—NH—, —CH₂—CH₂—O—CO—NH—; —CH₂—NH—CO—NH— or —CH₂—CH₂—NH—CO—NH—, —CH₂—CHOH—, —CH₂—CH₂—CHOH—, —CH₂—CH₂—CH(NH₂)—, —CH₂—CH(NH₂)—, —CH₂—CH₂—CH(NHR′)-, —CH₂—CH(NHR′)-, —CH₂—CH₂—CH(NR′R″)-, —CH₂—CH(NR′R″)-, —CH₂—CH₂—CH₂—NR′—, —CH₂—CH₂—CH₂—O—; —[CH₂—CH₂—O]ₙ— and —[CH₂—CH(CH₃)—O]ₙ—, —CH₂—CH₂—CHR′—O— with R′ and R″ representing a linear or branched C1-C22 alkyl optionally comprising 1 to 12 heteroatoms chosen from O, N, S, F, Si and P;
or a mixture of these radicals;
n′ is between 1 and 100 and preferably between 1 and 5 inclusive.

In formula (IId), the meaning of the radicals R1, Z′, x′, R′2 and m′ is the same as that given above for formula (IIa), and that of the radicals R′3 and n′ is the same as that given for formula (IIc).

In formula (IId), X″⁺ is a group of formula —N⁺R₆R₇R₈ with R6, R7 and R8 representing, independently of each other, either (i) a hydrogen atom, or (ii) a linear, branched or cyclic, optionally aromatic alkyl group containing from 1 to 18 carbon atoms, possibly comprising 1 to 5 heteroatoms chosen from O, N, S and P; or (iii) R6 and R7 may form with the nitrogen atom a first saturated or unsaturated, optionally aromatic ring comprising in total 5, 6 or 7 atoms, and especially 4, 5 or 6 carbon atoms and/or 2 to 3 heteroatoms chosen from O, S and N; said first ring possibly being fused with one or more other saturated or unsaturated, optionally aromatic rings, each comprising 5, 6 or 7 atoms, and especially 4, 5, 6 or 7 carbon atoms and/or 2 to 3 heteroatoms chosen from O, S and N.

For example, R6, R7 and R8 may be chosen from hydrogen and a methyl, ethyl, propyl, isopropyl, n-butyl, t-butyl, isobutyl, octyl, lauryl or stearyl group.

Among the preferred radicals X″⁺ that may be mentioned are trimethylammonium; triethylammonium; N,N-di-methyl-N-octylammonium; N,N-dimethyl-N-laurylammonium radicals.

Among the preferred monomers of formula (IIc) or (IId) that may be mentioned are N,N-dimethyl-N-(2-methacryloyloxyethyl)-N-(3-sulfopropyl)ammonium betaine (especially SPE from the company Raschig); N,N-dimethyl-N-(3-methacrylamidopropyl)-N-(3-sulfo-propyl)ammonium betaine (SPP from Raschig) and 1-(3-sulfopropyl)-2-vinylpyridinium betaine (SPV from Raschig), and also 2-methacryloyloxyethylphosphoryl-choline.

When the “essentially cationic” monomer is chosen from mixtures of cationic and/or amphoteric monomers with anionic monomers, said anionic monomers are preferably present in a proportion of from 5% to 40% by weight, especially from 10% to 30% by weight and preferably from 15% to 25% by weight relative to the weight of the “cationic and/or amphoteric+anionic” mixture.

The “essentially cationic” monomer is present in a proportion of from 40% to 90% by weight, especially from 45% to 80% by weight and preferably from 50% to 70% by weight relative to the weight of the final polymer.

Preferably, the polymer according to the invention comprises the monomers of formula (I) and the ionic monomers (the cationic monomers+the optional amphoteric and anionic monomers) in a weight ratio that may range from 60/40 to 40/60, with a preference for a 50/50 ratio.

The ethylenic copolymer according to the invention may optionally comprise monomers other than those mentioned above. These additional monomers are thus nonionic.

When it comprises such additional monomers, they are necessarily chosen from the monomers that are “hydrophilic” within the meaning of the present invention.

The term “hydrophilic monomer” means monomers with a value of the logarithm of the 1-octanol/water apparent partition coefficient, also known as the log P, of less than or equal to 2, for example between −8 and 2, preferably less than or equal to 1.5, especially less than or equal to 1 and in particular between −7 and 1, or even between −6 and 0.

The log P values are known and determined according to a standard test that determines the concentration of the monomer in 1-octanol and water.

The values may especially be calculated using the ACD software (Advanced Chemistry Development) software solaris V4.67; they may also be obtained from Exploring QSAR: hydrophobic, electronic and stearic constants (ACS professional reference book, 1995).

A website also exists that provides estimated values (address: http://esc.syrres.com/interkow/kowdemo.htm).

The log P value of certain common monomers, determined using the ACD software, are given below:

| | Methacrylate | Acrylate |
|---|---|---|
| | (* or meth- | (* or |
| | acrylamide) | acrylamide) |
| Methyl (meth)acrylate | 1.346 +/− 0.250 | 0.793 +/− 0.223 |
| Ethyl (meth)acrylate | 1.877 +/− 0.250 | 1.325 +/− 0.223 |
| Propyl (meth)acrylate | 2.408 +/− 0.250 | 1.856 +/− 0.223 |
| Isopropyl (meth)acrylate | 2.224 +/− 0.254 | 1.672 +/− 0.228 |
| Butyl (meth)acrylate | 2.940 +/− 0.250 | 2.387 +/− 0.223 |
| Isobutyl (meth)acrylate | 2.756 +/− 0.254 | 2.208 +/− 0.228 |
| Tert-butyl (meth)acrylate | 2.574 +/− 0.261 | 2.022 +/− 0.238 |
| Cyclohexyl (meth)acrylate | 3.405 +/− 0.252 | 2.853 +/− 0.226 |
| Octyl (meth)acrylate | 5.065 +/− 0.521 | 4.513 +/− 0.224 |
| Lauryl (meth)acrylate | 7.190 +/− 0.251 | 6.638 +/− 0.224 |
| Tridecyl (meth)acrylate | 7.712 +/− 0.251 | 7.170 +/− 0.224 |
| Cetyl (meth)acrylate | 9.316 +/− 0.251 | 8.764 +/− 0.224 |
| Palmityl (meth)acrylate | >9 | >9 |
| Stearyl (meth)acrylate | 10.379 +/− 0.251 | 9.826 +/− 0.224 |
| Behenyl (meth)acrylate | 11.952 +/− 0.225 | 12.504 ± 0.251 |
| Oleyl (meth)acrylate | >9 | 9.308 ± 0.232 |
| Tetrahydrofurfuryl | 1.352 ± 0.283 | 0.800 ± 0.263 |
| (meth)acrylate | | |
| 2-Ethyl hexyl | 4.881 ± 0.254 | 4.329 ± 0.229 |
| (meth)acrylate | | |
| 2-Hydroxyethyl | 0.718 ± 0.277 | 0.166 ± 0.258 |
| (meth)acrylate | | |
| Ethoxyethyl | 1.887 ± 0.293 | 1.335 ± 0.268 |
| (meth)acrylate | | |
| Hydroxypropyl | | 0.383 ± 0.241 |
| (meth)acrylate | | |
| N-isopropyl | 0.748 ± 0.276 | 0.195 ± 0.256 |
| (meth)acrylamide* | | |
| N-octyl (meth)acrylamide* | 3.558 ± 0.273 | 3.036 ± 0.253 |
| N,N-dimethyl | 0.906 ± 0.553 | −0.168 ± 0.556 |
| (meth)acrylamide* | | |
| N,N-dibutyl | 3.573 ± 0.570 | 3.021 ± 0.557 |
| (meth)acrylamide* | | |

| | |
|---|---|
| Vinyl acetate | 0.730 ± 0.286 |
| Methyl vinyl ether | 0.509 ± 0.286 |
| Ethyl vinyl ether | 1.040 ± 0.286 |
| Vinylcaprolactam | 1.499 ± 0.207 |
| Vinylpyrrolidone | 0.370 ± 0.206 |
| N-vinylacetamide | 0 ± 0.231 |

The additional hydrophilic monomers may be chosen especially from those of formula (III), alone or as a mixture:

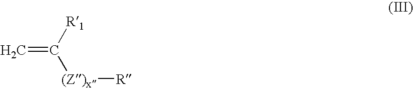

in which:
R′₁ is hydrogen or —CH₃;
Z″ is a divalent group chosen from —COO—, —CONH—, —CONCH₃—, —OCO—, —SO₂, —CO—O—CO—, —CO—CH₂—CO— and —O—; preferably COO and CONH;
x″ is 0 or 1;
R″ is a saturated or unsaturated, optionally aromatic, linear, branched or cyclic carbon-based radical of 1 to 30 carbon atoms, possibly comprising 1 to 18 heteroatoms chosen from O, N, S, F, Si and P.
In the radical R″, the heteroatom(s), when they are present, may be intercalated in the chain of said radical, or alternatively said radical may be substituted with one or more groups comprising them, such as hydroxyl, ester, amide, urethane or urea.
R″ may especially be a methyl, ethyl, propyl, isopropyl, n-butyl, isobutyl, tert-butyl, phenyl or benzyl radical, or a radical of formula —CH₂—CH₂—CH₂OH, —CH₂—CH₂—OH, —CH₂—CH₂—CH₂OH or furfuryl.

The additional hydrophilic nonionic monomers are especially chosen from the following monomers: methyl methacrylate, methyl acrylate, ethyl methacrylate, ethyl acrylate, propyl acrylate, isopropyl acrylate, tetrahydrofurfuryl methacrylate, tetrahydrofurfuryl acrylate, 2-hydroxyethyl methacrylate, 2-hydroxyethyl acrylate, ethoxyethyl methacrylate, ethoxy-ethylacrylate, N-isopropylacrylamide, N-isopropylmeth-acrylamide, N,N-dimethylacrylamide, N,N-dimethylmeth-acrylamide, vinyl acetate, methyl vinyl ether, ethyl vinyl ether, vinylpyrrolidone, vinylcaprolactam, N-vinylacetamide, hydroxylpropyl acrylate, N-vinyllactam, acrylamide, N-methylacrylamide, N,N-dimethylacrylamide, N-methyl-N-vinylacetamide, N-vinylformamide, N-methyl-N-vinylformamide, vinyl alcohol (copolymerized in the form of vinylacetate and then hydrolyzed).

The additional monomer, alone or as a mixture, may not be present in the polymer according to the invention (0%), or alternatively may be present in an amount that may range up to 50% by weight, relative to the weight of the final polymer; it may especially be present in an amount of from 0.1% to 35% by weight, preferably from 1% to 25% by weight, for example from 3% to 15% by weight, or even from 5% to 9.5% by weight, relative to the total weight of the polymer.

However, it has been found that when this additional monomer is chosen from methyl acrylate, methyl methacrylate and isopropyl acrylate, these monomers could not be present in an amount of greater than or equal to 10% by weight. These monomers may thus be present in a proportion of 0-9.5% by weight, especially from 0.1% to 8% by weight and preferably from 1% to 5% by weight in the final polymer.

Preferably, the copolymer according to the invention does not comprise any monomers other than those of formulae (I), (IIa), (IIb), (IIc) and (IId). Preferably, the copolymer according to the invention comprises only monomers of formulae (I) and (IIa).

Preferably, the polymer that may be used according to the invention comprises the monomers of formula (I) and the “essentially cationic” monomers in a weight ratio that may range from 60/40 to 40/60, with a preference for a 50/50 ratio.

In one particular embodiment of the invention, the polymer consists essentially of monomer of formula (I), alone or as a mixture, and of monomers of formula (IIa), alone or as a mixture.

The polymers that are most particularly preferred are those in which:
the monomer of formula (I), alone or as a mixture, is present in a proportion of from 10% to 60% by weight, especially from 20% to 60% by weight and preferably from 30% to 50% by weight, relative to the weight of the final polymer, and is chosen, alone or as a mixture, from poly(ethylene glycol) (meth)acrylates, preferably those with a molecular weight of between 350 and 13 000 g/mol and especially between 500 and 8000 g/mol; and
the “essentially cationic” monomer is present in a proportion of from 40% to 90% by weight, especially from 40% to 80% by weight and preferably from 50% to 70% by weight relative to the weight of the final polymer, and is chosen, alone or as a mixture, from dimethylaminopropyl (meth)acrylamide, dimethyl-aminoethyl (meth)acrylamide, diethylaminoethyl (meth)acrylate, dimethylaminoethyl (meth)acrylate, vinylimidazole, vinylpyridine and morpholinoethyl (meth)acrylate; and
the polymer preferably being neutralized with an organic acid neutralizer chosen especially from 2-ethylcaproic acid, oleic acid, behenic acid, stearic acid, acetic acid, citric acid, tartaric acid, betaine hydrochloride and/or gluconic acid, and preferentially behenic acid and/or betaine hydrochloride.

The polymers that are even more particularly preferred are those in which:
the monomer of formula (I), alone or as a mixture, is present in a proportion of from 10% to 60% by weight, especially from 20% to 60% by weight and preferably from 30% to 50% by weight, relative to the weight of the final polymer, and is chosen, alone or as a mixture, from poly(ethylene glycol) (meth)acrylates, preferably those with a molecular weight of between 350 and 13 000 g/mol and especially between 500 and 8000 g/mol; and
the “essentially cationic” monomer is present in a proportion of from 40% to 90% by weight, especially from 40% to 80% by weight and preferably from 50% to 70% by weight, relative to the weight of the final polymer, and is chosen, alone or as a mixture, from dimethylaminopropyl (meth)acrylamide, and
the polymer being neutralized with a neutralizer chosen from behenic acid and/or betaine hydrochloride.

The polymers according to the invention may be prepared according to the usual standard radical polymerization methods known to those skilled in the art, and as described, for example, in the book “Chimie et physicochimie des polyméres” by Gnanou et al. (published by Dunod).

These polymers may especially be prepared by:
direct solution polymerization in water with optional preneutralization of the cationic unit and/or of the anionic unit;
emulsion polymerization in water with optional preneutralization of the cationic unit and/or of the anionic unit, with use of a surfactant;
polymerization in an organic solvent, such as ethanol or methyl ethyl ketone, with optional preneutralization of the cationic unit and/or of the anionic unit, followed by a step of dissolution or dispersion in water with evaporation of the solvent.

These polymerizations may be performed in the presence of a radical initiator especially of peroxide type (Trigonox 21S: tert-butyl peroxy-2-ethylhexanoate) or azo type (AIBN V50: 2,2′-azobis(2-amidinopropane) dihydrochloride), which may be present in a proportion of from 0.3% to 5% by weight relative to the total weight of the monomers.

The polymers according to the invention are noncrosslinked. They are in the form of statistical, preferably film-forming, ethylenic copolymers of one more ethylenic monomers containing PEG groups (the PEG groups are pendent along the backbone) and of one or more ethylenic monomers comprising cationic functions (nonquaternary neutralized amines) and/or betaine functions and, optionally, one or more other monovalent nonionic hydrophilic ethylenic comonomers.

The term “ethylenic” polymer means a polymer obtained by polymerization of ethylenically unsaturated monomers.

The term “film-forming” polymer means a polymer that can form, by itself or in the presence of an auxiliary film-forming agent, a continuous film that adheres to a support, especially to keratin materials.

They have a weight-average molecular mass (Mw) that is preferably between 500 and 5 000 000, especially between 1000 and 3 000 000 and more preferentially between 2000 and 2 000 000, or even between 4000 and 500 000, better still between 7000 and 250 000 and even better between 8000 and 100 000.

The weight-average molar masses (Mw) are determined by gel permeation chromatography or by light scattering, depending on the accessibility of the method (solubility of the polymers under consideration).

The polymers that may be used according to the invention may preferably be conveyed in aqueous medium, i.e. they are preferably water-soluble or water-dispersible.

The term “water-soluble” means that it is soluble in water, to a proportion of at least 5% by weight, at 25° C., and forms a clear solution.

The term “water-dispersible” means that it forms in water, at a concentration of 5% by weight, at 25° C., a stable dispersion of fine, generally spherical particles. The mean size of the particles constituting said dispersion is less than 1 μm and more generally ranges between 5 and 400 nm and preferably from 10 to 250 nm. These particle sizes are measured by light scattering.

The dissolution or dispersion in water may be performed by direct dissolution of the polymer if it is soluble, or alternatively by neutralization of the amine and/or acid units so as to make the polymer soluble or dispersible in water. The dissolution or dispersion in water may also be performed via an intermediate step of dissolution in an organic solvent followed by the addition of water before evaporation of the organic solvent.

Moreover, it has been found that the polymers that may be used according to the invention advantageously have a viscosity in water that is adequate for the envisioned applications, which may be, for example, between 1 and 1000 mPa·s, preferably between 1.5 and 750 mPa·s and better still between 2 and 500 mPa·s.

The viscosity is measured using a Brookfield viscometer, for a solution containing 15% by weight of polymer in water or methyl ethyl ketone (solvent chosen as a function of the solubility of the polymer and/or of the polymerization method), at 25° C., with a needle-type spindle chosen from the model numbers 00 to 07 from Brookfield, preferably a No. 1 spindle; for a measuring time of 5 minutes, at a speed of between 0.1 and 6 rpm. The viscosity is measured after total dissolution of the polymer in water or methyl ethyl ketone.

In addition, the polymers that may be used according to the invention may preferably have a glass transition temperature (Tg) of between −150° C. and 20° C., especially−120° C. and 10° C. and better still between −100° C. and 0° C.; the Tg is measured according to the method given before the examples.

The polymers that may be used according to the invention may preferably have a melting point (m.p.) of between −100° C. and 80° C., especially between −80° C. and 50° C. and better still between −70° C. and 45° C., or even between −10° C. and 25° C.

In addition, the polymers that may be used according to the invention preferably have a water uptake of between 3% and 150% by weight, preferably between 4% and 100% by weight and especially between 5% and 50% by weight, at 75% relative humidity (75% HR); the water uptake is measured according to the method given before the examples.

They may also have a water uptake of between 3% and 20% by weight, preferably between 2.5% and 150% by weight and especially between 3% and 100% by weight, at 85% relative humidity (85% HR).

The polymers may be present in the composition in dissolved form, for example in water or an organic solvent, or alternatively in the form of an aqueous or organic dispersion.

They may be used in the cosmetic compositions according to the invention in a proportion of from 0.01% to 30% by weight of solids, especially from 0.1% to 20% by weight or even from 0.1% to 10% by weight and better still from 0.5% to 3% by weight relative to the total weight of the composition.

The composition may thus comprise a hydrophilic medium comprising water or a mixture of water and of hydrophilic organic solvent(s), for instance alcohols and especially linear or branched C₁-C₆ lower monoalcohols, for instance, ethanol, tert-butanol, n-butanol, isopropanol or n-propanol, and polyols, for instance glycerol, diglycerol, propylene glycol, sorbitol, pentylene glycol and polyethylene glycols, or alternatively glycol ethers, especially C₂ glycol ethers, and hydrophilic C₂-C₄ ketones.

Water or a mixture of water and of hydrophilic organic solvents may be present in the composition according to the invention in an amount ranging from 30% to 99% by weight, and preferably from 40% to 80% by weight, relative to the total weight of the composition.

The composition may also comprise a fatty phase consisting especially of fatty substances that are liquid at room temperature (in general 25° C.) and/or fatty substances that are solid at room temperature, such as waxes, pasty fatty substances and gums, and mixtures thereof. These fatty substances may be of animal, plant, mineral or synthetic origin. This fatty phase may also contain lipophilic organic solvents.

As fatty substances that are liquid at room temperature, often known as oils, which may be used in the invention, mention may be made of: hydrocarbon-based oils of animal origin such as perhydrosqualene; hydrocarbon-based plant oils such as liquid triglycerides of fatty acids containing from 4 to 10 carbon atoms, for instance heptanoic or octanoic acid triglyceride, or alternatively sweet almond oil, olive oil, wheatgerm oil, groundnut oil, rapeseed oil, safflower oil, coconut oil, hazlenut oil, palm oil, apricot kernel oil, calophyllum oil, sunflower oil, corn oil, soybean oil, grapeseed oil, sesame seed oil, macadamia oil, castor oil, avocado oil, caprylic/capric acid triglycerides, jojoba oil and shea butter; linear or branched hydrocarbons of mineral or synthetic origin such as liquid paraffins and derivatives thereof, petroleum jelly, polydecenes and hydrogenated polyisobutene such as parleam; synthetic esters and synthetic ethers, especially of fatty acids, such as, for example, purcellin oil, isopropyl myristate, 2-ethylhexyl palmitate, 2-octyldodecyl stearate, 2-octyldodecyl erucate and isostearyl isostearate; hydroxylated esters, for instance isostearyl lactate, octyl hydroxystearate, octyldodecyl hydroxystearate, diisostearyl malate, triisocetyl citrate, and fatty alkyl heptanoates, octanoates and decanoates; polyol esters, for instance propylene glycol dioctanoate, neopentyl glycol diheptanoate or diethylene glycol diisononanoate; and pentaerythritol esters; fatty alcohols containing from 12 to 26 carbon atoms, for instance octyldodecanol, 2-butyloctanol, 2-hexyldecanol, 2-undecylpentadecanol and oleyl alcohol; partially hydrocarbon-based or silicone-based fluoro oils; silicone-based oils; mixtures thereof.

These oils may be present in a content ranging from 0.01% to 90% and better still from 0.1% to 85% by weight, relative to the total weight of the composition.

For the purposes of the present invention, the term “wax” means a lipophilic compound that is solid at room temperature (25° C.), which undergoes a reversible solid/liquid change of state, and which has a melting point of greater than or equal to 25° C., which may be up to 120° C. By bringing the wax to the liquid state (melting), it is possible to make it miscible with the oils possibly present and to form a microscopically homogeneous mixture, but, on returning the temperature of the mixture to room temperature, recrystallization of the wax is obtained in the oils of the mixture. The melting point of the wax may be measured using a differential scanning calorimeter (DSC), for example the calorimeter sold under the name DSC 30 by the company Mettler.

The waxes may be hydrocarbon-based waxes, fluoro waxes and/or silicone waxes and may be of plant, mineral, animal and/or synthetic origin. In particular, the waxes have a melting point of greater than 30° C. and preferably greater than 45° C. As waxes that may be used in the composition of the invention, mention may be made of beeswax, carnauba wax or candelilla wax, paraffin, microcrystalline waxes, ceresin or ozokerite; synthetic waxes, for instance polyethylene waxes or Fischer-Tropsch waxes, or silicone waxes, for instance alkyl dimethicones or alkoxy dimethicones containing from 16 to 45 carbon atoms.

The gums are generally polydimethylsiloxanes (PDMSs) of high molecular weight or cellulose gums or polysaccharides and the pasty substances are generally hydrocarbon-based compounds, for instance lanolins and derivatives thereof, or PDMSs.

The nature and amount of the solid substances depend on the desired mechanical properties and textures. As a guide, the composition may contain from 0.1 to 50% by weight and better still from 1% to 30% by weight of waxes, relative to the total weight of the composition.

The composition may also comprise an additional polymer such as a film-forming polymer. According to the present invention, the term “film-forming polymer” means a polymer that is capable, by itself or in the presence of an auxiliary film-forming agent, of forming a continuous film that adheres to a support, especially to keratin materials. Among the film-forming polymers that may be used in the composition of the present invention, mention may be made of synthetic polymers, of radical type or of polycondensate type, and polymers of natural origin, and mixtures thereof, in particular acrylic polymers, polyurethanes, polyesters, polyamides, polyureas and cellulose-based polymers such as nitrocellulose.

The composition may also comprise a conditioning polymer other than the copolymers with a PEG graft, generally consisting of a cationic polymer.

The cationic polymers that may be used in accordance with the present invention may be chosen from any of those already known per se as improving the cosmetic properties of the hair, i.e. especially those described in patent application EP-A-0 337 354 and in French patent applications FR-A-2 270 846, 2 383 660, 2 598 611, 2 470 596 and 2 519 863 and having a suitable cationic charge density.

Even more generally, for the purposes of the present invention, the expression “cationic polymer” denotes any polymer containing cationic groups and/or groups that may be ionized into cationic groups.

The cationic polymers that are preferred are chosen from those containing units comprising primary, secondary, tertiary and/or quaternary amine groups that may either form part of the main polymer chain, or be borne by a side substituent that is directly attached to said chain.

The cationic polymers used generally have a number-average or weight-average molar mass of between 500 and 5×10⁶ approximately and preferably between 10³ and 3×10⁶ approximately.

Among the cationic polymers that may be mentioned more particularly are polymers of the polyamine, polyaminoamide and polyquaternary ammonium type. These are known products.

The polymers of the polyamine, polyaminoamide and polyquaternary ammonium type that may be used in accordance with the present invention, and that may especially be mentioned, are those described in French patents 2 505 348 or 2 542 997. Among these polymers, mention may be made of:

(1) homopolymers or copolymers derived from acrylic or methacrylic esters or amides and comprising at least one of the units of the following formulae:

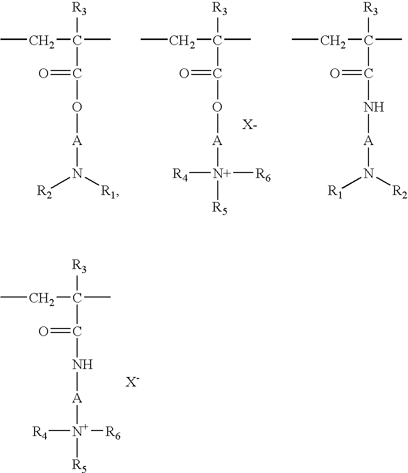

in which:
R₃, which may be identical or different, denote a hydrogen atom or a CH₃ radical;
A, which may be identical or different, represent a linear or branched alkyl group of 1 to 6 carbon atoms, preferably 2 or 3 carbon atoms, or a hydroxyalkyl group of 1 to 4 carbon atoms;
R₄, R₅ and R₆, which may be identical or different, represent an alkyl group containing from 1 to 18 carbon atoms or a benzyl radical and preferably an alkyl group containing from 1 to 6 carbon atoms;
R₁ and R₂, which may be identical or different, represent hydrogen or an alkyl group containing from 1 to 6 carbon atoms, and preferably methyl or ethyl;
X denotes an anion derived from an inorganic or organic acid, such as a methosulfate anion or a halide such as chloride or bromide.

Copolymers of family (1) can also contain one or more units derived from comonomers which may be chosen from the family of acrylamides, methacrylamides, diacetone acrylamides, acrylamides and methacrylamides substituted on the nitrogen with lower (C₁-C₄) alkyls, acrylic or methacrylic acids or esters thereof, vinyllactams such as vinylpyrrolidone or vinylcaprolactam, and vinyl esters.

Thus, among these copolymers of family (1), mention may be made of:
copolymers of acrylamide and of dimethylaminoethyl methacrylate quaternized with dimethyl sulfate or with a dimethyl halide, such as the product sold under the name Hercofloc by the company Hercules,
the copolymers of acrylamide and of methacryloyloxy-ethyltrimethylammonium chloride described, for example, in patent application EP-A-080 976 and sold under the name Bina Quat P 100 by the company Ciba Geigy,
the copolymers of acrylamide and of methacryloyloxy-ethyltrimethylammonium methosulfate sold under the name Reten by the company Hercules,
quaternized or nonquaternized vinylpyrrolidone/dialkylaminoalkyl acrylate or methacrylate copolymers.

These polymers are described in detail in French patents 2 077 143 and 2 393 573,
dimethylaminoethyl methacrylate/vinylcaprolactam/vinylpyrrolidone terpolymers,
vinylpyrrolidone/methacrylamidopropyldimethylamine copolymers, and
quaternized vinylpyrrolidone/dimethylaminopropyl-methacrylamide copolymers.

(2) cationic polysaccharides, especially celluloses and cationic galactomannan gums. Among the cationic polysaccharides that may be mentioned more particularly are cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups, which are described in French patent 1 492 597. These polymers are also defined in the CTFA dictionary as hydroxyethylcellulose quaternary ammoniums that have reacted with an epoxide substituted with a trimethylammonium group.

The cationic cellulose copolymers or cellulose derivatives grafted with a water-soluble quaternary ammonium monomer are described especially in patent U.S. Pat. No. 4,131,576, such as hydroxyalkylcelluloses, for instance hydroxymethyl-, hydroxyethyl- or hydroxy-propylcelluloses grafted especially with a methacryl-oylethyltrimethylammonium, methacrylamidopropyltrimeth-ylammonium or dimethyldiallylammonium salt.

The cationic galactomannan gums are described more particularly in U.S. Pat. Nos. 3,589,578 and 4,031,307, in particular guar gums containing trialkylammonium cationic groups. Use is made, for example, of guar gums modified with a salt (e.g. chloride) of 2,3-epoxy-propyltrimethylammonium.

(3) polymers consisting of piperazinyl units and of divalent alkylene or hydroxyalkylene radicals containing straight or branched chains, optionally interrupted by oxygen, sulfur or nitrogen atoms or by aromatic or heterocyclic rings, as well as the oxidation and/or quaternization products of these polymers. Such polymers are described, in particular, in French patents 2 162 025 and 2 280 361;

(4) water-soluble polyamino amides prepared in particular by polycondensation of an acidic compound with a polyamine; these polyamino amides can be crosslinked with an epihalohydrin, a diepoxide, a dianhydride, an unsaturated dianhydride, a bis-unsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent is used in proportions ranging from 0.025 to 0.35 mol per amine group of the polyamino amide; these polyamino amides can be alkylated or, if they contain one or more tertiary amine functions, they can be quaternized. Such polymers are described, in particular, in French patents 2 252 840 and 2 368 508;

(5) polyaminoamide derivatives resulting from the condensation of polyalkylene polyamines with polycarboxylic acids followed by alkylation with difunctional agents. Mention may be made, for example, of adipic acid/dialkylaminohydroxyalkyldialkylene-triamine polymers in which the alkyl radical contains from 1 to 4 carbon atoms and preferably denotes methyl, ethyl or propyl. Such polymers are described in particular in French patent 1 583 363.

Among these derivatives, mention may be made more particularly of the adipic acid/dimethylamino-hydroxypropyl/diethylenetriamine polymers sold under the name “Cartaretine F, F4 or F8” by the company Sandoz.

(6) polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The molar ratio between the polyalkylene polyamine and the dicarboxylic acid is between 0.8:1 and 1.4:1; the polyamino amide resulting therefrom is reacted with epichlorohydrin in a molar ratio of epichlorohydrin relative to the secondary amine group of the polyaminoamide of between 0.5:1 and 1.8:1. Such polymers are described in particular in U.S. Pat. Nos. 3,227,615 and 2,961,347.

Polymers of this type are sold in particular under the name “Hercosett 57” by the company Hercules Inc. by the company Hercules in the case of the adipic acid/epoxypropyl/diethylenetriamine copolymer.

(7) cocyclopolymers of alkyldiallylamine or of dialkyldiallylammonium, such as the copolymers containing, as main constituent of the chain, units corresponding to formula (I) or (I′):

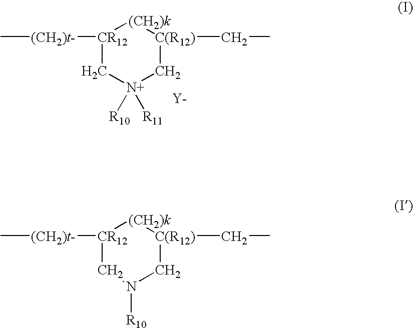

in which formulae k and t are equal to 0 or 1, the sum k+t being equal to 1; R₁₂ denotes a hydrogen atom or a methyl radical; R₁₀ and R₁₁, independently of each other, denote an alkyl group having from 1 to 6 carbon atoms, a hydroxyalkyl group in which the alkyl group preferably has 1 to 5 carbon atoms, a lower C₁-C₄ amidoalkyl group, or R₁₀ and R₁₁ can denote, together with the nitrogen atom to which they are attached, heterocyclic groups such as piperidyl or morpholinyl; Y⁻ is an anion such as bromide, chloride, acetate, borate, citrate, tartrate, bisulfate, bisulfite, sulfate or phosphate. These polymers are described in particular in French patent 2 080 759 and in its Certificate of Addition 2 190 406.

R₁₀ and R₁₁, independently of each other, preferably denote an alkyl group containing from 1 to 4 carbon atoms.

Among the polymers defined above, mention may be made more particularly of the dimethyldiallylammonium chloride homopolymer sold under the name “Merquat 100” by the company Nalco (and the homologs thereof with low weight-average molar masses) and copolymers of diallyldimethylammonium chloride and acrylamide chloride.

(8) quaternary diammonium polymers containing repeating units corresponding to the formula:

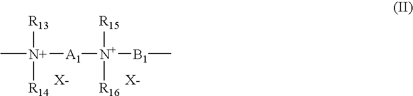

in which formula (II):

R₁₃, R₁₄, R₁₅ and R₁₆, which may be identical or different, represent aliphatic, alicyclic or arylaliphatic radicals containing from 1 to 20 carbon atoms or lower hydroxyalkylaliphatic radicals, or alternatively R₁₃, R₁₄, R₁₅ and R₁₆, together or separately, constitute, with the nitrogen atoms to which they are attached, heterocycles optionally containing a second hetero atom other than nitrogen, or alternatively R₁₃, R₁₄, R₁₅ and R₁₆ represent a linear or branched C₁-C₆ alkyl radical substituted with a nitrile, ester, acyl or amide group or a group —CO—O—R₁₇-D or —CO—NH—R₁₇-D where R₁₇ is an alkylene and D is a quaternary ammonium group;

A₁ and B₁ represent polymethylene groups containing from 2 to 20 carbon atoms, which groups may be linear or branched, saturated or unsaturated, and possibly containing, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and

X⁻ denotes an anion derived from an inorganic or organic acid;

A₁, R₁₃ and R₁₅ can form, with the two nitrogen atoms to which they are attached, a piperazine ring; in addition, if A₁ denotes a linear or branched, saturated or unsaturated alkylene or hydroxyalkylene radical, B₁ can also denote a group (CH₂)ₙ—CO-D-OC—(CH₂)ₙ—

in which D denotes:
a) a glycol residue of formula: —O-Z—O—, where Z denotes a linear or branched hydrocarbon-based radical or a group corresponding to one of the following formulae:

—(CH₂—CH₂—O)ₓ—CH₂—CH₂—

—[CH₂—CH(CH₃)—O]_{y}—CH₂—CH(CH₃)—

where x and y denote an integer from 1 to 4, representing a defined and unique degree of polymerization or any number from 1 to 4 representing an average degree of polymerization;
b) a bis-secondary diamine residue such as a piperazine derivative;
c) a bis-primary diamine residue of formula: —NH—Y—NH—, where Y denotes a linear or branched hydrocarbon-based radical, or alternatively the divalent radical

—CH₂—CH₂—S—S—CH₂—CH₂—;
d) a ureylene group of formula: —NH—CO—NH—;

Preferably, X⁻ is an anion such as chloride or bromide.

These polymers generally have a number-average molecular mass of between 1000 and 100 000.

Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027, 020.

It is more particularly possible to use polymers that consist of repeating units corresponding to the formula:

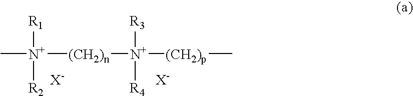

in which R₁, R₂, R₃ and R₄, which may be identical or different, denote an alkyl or hydroxyalkyl radical containing from 1 to 4 carbon atoms approximately, n and p are integers ranging from 2 to 20 approximately, and X⁻ is an anion derived from an inorganic or organic acid.

One compound of formula (a) that is particularly preferred is the one for which R₁, R₂, R₃ and R₄ represent a methyl radical and n=3, p=6 and X═Cl, which is known as Hexadimethrine chloride according to the INCI (CTFA) nomenclature.

(9) polyquaternary ammonium polymers consisting of units of formula (III):

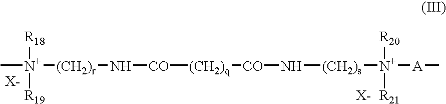

in which formula:
R₁₈, R₁₉, R₂₀ and R₂₁, which may be identical or different, represent a hydrogen atom or a methyl, ethyl, propyl, β-hydroxyethyl, β-hydroxypropyl or —CH₂CH₂ (OCH₂CH₂)ₚOH radical,
where p is equal to 0 or to an integer between 1 and 6, with the proviso that R₁₈, R₁₉, R₂₀ and R₂₁ do not simultaneously represent a hydrogen atom,
r and s, which may be identical or different, are integers between 1 and 6,
q is equal to 0 or to an integer between 1 and 34,
X⁻ denotes an anion such as a halide,
A denotes a dihalide radical or preferably represents —CH₂—CH₂—O—CH₂—CH₂—.

Such compounds are described in particular in patent application EP-A-122 324.

Among these products, mention may be made, for example, of “Mirapol® A 15”, “Mirapol® AD1”, “Mirapol® AZ1” and “Mirapol® 175” sold by the company Miranol.

(10) quaternary polymers of vinylpyrrolidone and of vinylimidazole, such as, for example, the products sold under the names Luviquat® FC 905, FC 550 and FC 370 by the company BASF.

(11) crosslinked methacryloyloxy(C₁-C₄)alkyltri (C₁-C₄)-alkylammonium salt polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound containing olefinic unsaturation, in particular methylenebisacrylamide.

Other cationic polymers that can be used in the context of the invention are cationic proteins or cationic protein hydrolysates, polyalkyleneimines, in particular polyethyleneimines, polymers containing vinylpyridine or vinylpyridinium units, condensates of polyamines and of epichlorohydrin, quaternary polyureylenes and chitin derivatives, especially chitosans or salts thereof;

The salts that may be used are in particular chitosan acetate, chitosan lactate, chitosan glutamate, chitosan gluconate or chitosan pyrrolidonecarboxylate.

Among these compounds, mention may be made of chitosans with a degree of deacetylation of 90% by weight and chitosan pyrrolidonecarboxylate sold under the name Kytamer® PC by the company Amerchol.

Among all the cationic polymers that may be used in the context of the present invention, it is preferred to use cationic cyclopolymers, in particular the dimethyldiallylammonium chloride homopolymers or copolymers sold under the names “Merquat 100”, “Merquat 550” and “Merquat S” by the company Nalco, quaternary polymers of vinylpyrrolidone and of vinyl-imidazole, crosslinked homopolymers or copolymers of methacryloyloxy(C₁-C₄)alkyltri (C₁-C₄)alkylammonium salts, and chitosan pyrrolidonecarboxylate sold under the name Kytamer® PC by the company Amerchol, and mixtures thereof.

According to the invention, the cationic or amphoteric polymer(s) may represent from 0.001% to 20% by weight, preferably from 0.01% to 10% by weight and more particularly from 0.02% to 5% by weight relative to the total weight of the final composition.

According to one preferred embodiment of the invention, the compositions may also comprise at least one silicone.

As silicones that may be used in the compositions of the present invention, mention may be made in particular of the volatile or nonvolatile, cyclic or acyclic, branched or unbranched, organomodified or non-organomodified silicones as described below. The silicones that may be used in accordance with the invention may be soluble or insoluble in the composition and may in particular be polyorgano-siloxanes that are insoluble in the composition of the invention; they may be in the form of oils, waxes, resins or gums.

According to the invention, the silicones may all be used in unmodified form or in the form of solutions, dispersions, emulsions, nanoemulsions or micro-emulsions.

The organopolysiloxanes are defined in greater detail in Walter Noll's “Chemistry and Technology of Silicones” (1968) Academic Press. They can be volatile or nonvolatile.

When they are volatile, the silicones are more particularly chosen from those having a boiling point of between 60° C. and 260° C., and even more particularly from:

(i) cyclic silicones containing from 3 to 7 and preferably 4 to 5 silicon atoms. These are, for example, octamethylcyclotetrasiloxane sold in particular under the name Volatile Silicone 7207 by Union Carbide or Silbione 70045 V 2 by Rhodia, decamethylcyclopentasiloxane sold under the name Volatile Silicone 7158 by Union Carbide, and Silbione 70045 V 5 by Rhodia, and mixtures thereof.

Mention may also be made of cyclocopolymers of the dimethylsiloxanes/methylalkylsiloxane type, such as Volatile Silicone FZ 3109 sold by the company Union Carbide, having the chemical structure:

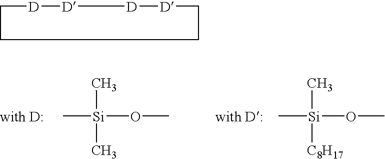

Mention may also be made of mixtures of cyclic silicones with organosilicon compounds, such as the mixture of octamethylcyclotetrasiloxane and tetra-trimethylsilylpentaerythritol (50/50) and the mixture of octamethylcyclotetrasiloxane and oxy-1,1′-bis(2,2,2′,2′,3,3′-hexatrimethylsilyloxy)neopentane;

(ii) linear volatile silicones containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, Jan. 76, pp. 27-32, Todd & Byers “Volatile Silicone Fluids for Cosmetics”.

Nonvolatile silicones, and more particularly poly-alkylsiloxanes, polyarylsiloxanes, polyalkylarylsiloxanes, silicone gums and resins, polyorganosiloxanes modified with organofunctional groups, and mixtures thereof, are preferably used.

These silicones are more particularly chosen from polyalkylsiloxanes, among which mention may be made mainly of polydimethylsiloxanes containing trimethyl-silyl end groups having a viscosity of from 5×10⁻⁶ to 2.5 m²/s at 25° C. and preferably 1×10⁻⁵ to 1 m²/s. The viscosity of the silicones is measured, for example, at 25° C. according to ASTM standard 445 Appendix C.

Among these polyalkylsiloxanes, mention may be made, in a nonlimiting manner, of the following commercial products:

the Silbione oils of the 47 and 70 047 series or the Mirasil oils sold by Rhodia, such as, for example, the oil 70 047 V 500 000;
the oils of the Mirasil series sold by the company Rhodia;
the oils of the 200 series from the company Dow Corning, such as, more particularly, DC200 with a viscosity of 60 000 mm²/s;
the Viscasil oils from General Electric and certain oils of the SF series (SF 96, SF 18) from General Electric.

Mention may also be made of polydimethylsiloxanes containing dimethylsilanol end groups, known under the name dimethiconol (CTFA name), such as the oils of the 48 series from the company Rhodia.

In this category of polyalkylsiloxanes, mention may also be made of the products sold under the names Abil Wax 9800 and 9801 by the company Goldschmidt, which are poly (C₁-C₂₀) alkylsiloxanes.

The polyalkylarylsiloxanes are chosen particularly from linear and/or branched polydimethylmethylphenyl-siloxanes and polydimethyldiphenylsiloxanes with a viscosity of from 1×10⁻⁵ to 5×10⁻² m²/s at 25° C.

Among these polyalkylarylsiloxanes, mention may be made, by way of example, of the products sold under the following names:
the Silbione oils of the 70 641 series from Rhodia;
the oils of the Rhodorsil 70 633 and 763 series from Rhodia;
the oil Dow Corning 556 Cosmetic Grade Fluid from Dow Corning;
the silicones of the PK series from Bayer, such as the product PK20;
the silicones of the PN and PH series from Bayer, such as the products PN1000 and PH1000;
certain oils of the SF series from General Electric, such as SF 1023, SF 1154, SF 1250 and SF 1265.

The silicone gums that can be used in accordance with the invention are, in particular, polydiorganosiloxanes with high number-average molecular masses of between 200 000 and 1 000 000, used alone or as a mixture in a solvent. This solvent can be chosen from volatile silicones, polydimethylsiloxane (PDMS) oils, poly-phenylmethylsiloxane (PPMS) oils, isoparaffins, polyisobutylenes, methylene chloride, pentane, dodecane and tridecane, or mixtures thereof.

Mention may be made more particularly of the following products:
polydimethylsiloxane,
polydimethylsiloxane/methylvinylsiloxane gums,
polydimethylsiloxane/diphenylsiloxane,
polydimethylsiloxane/phenylmethylsiloxane,
polydimethylsiloxane/diphenylsiloxane/methylvinyl-siloxane.

Products that can be used more particularly in accordance with the invention are mixtures such as:
mixtures formed from a polydimethylsiloxane hydroxylated at the end of the chain or (CTFA) and from a cyclic polydimethylsiloxane also called cyclomethicone (CTFA), such as the product Q2 1401 sold by the company Dow Corning;
mixtures formed from a polydimethylsiloxane gum with a cyclic silicone, such as the product SF 1214 Silicone Fluid from the company General Electric; this product is an SF 30 gum corresponding to a dimethicone, having a number-average molecular weight of 500 000, dissolved in the oil SF 1202 Silicone Fluid corresponding to decamethylcyclopentasiloxane;
mixtures of two PDMSs of different viscosities, and more particularly of a PDMS gum and a PDMS oil, such as the product SF 1236 from the company General Electric. The product SF 1236 is a mixture of an SE 30 gum defined above, having a viscosity of 20 m²/s; and an SF 96 oil, with a viscosity of 5×10⁻⁶ m²/s. This product preferably contains 15% SE 30 gum and 85% SF 96 oil.

The organopolysiloxane resins that can be used in accordance with the invention are crosslinked siloxane systems containing the following units:

R₂SiO_{2/2}, R₃SiO_{1/2}, RSiO_{3/2} and SiO_{4/2} in which R represents a hydrocarbon-based group containing 1 to 16 carbon atoms or a phenyl group. Among these products, those particularly preferred are the ones in which R denotes a C₁-C₄ lower alkyl group, more particularly methyl, or a phenyl group.

Among these resins, mention may be made of the product sold under the name Dow Corning 593 or those sold under the names Silicone Fluid SS 4230 and SS 4267 by the company General Electric, which are silicones of dimethyl/trimethylsiloxane structure.

Mention may also be made of the trimethyl siloxysilicate type resins sold in particular under the names X22-4914, X21-5034 and X21-5037 by the company Shin-Etsu.

The organomodified silicones that can be used in accordance with the invention are silicones as defined above and comprising in their structure one or more organofunctional groups attached via a hydrocarbon-based radical.

Among the organomodified silicones, mention may be made of polyorganosiloxanes comprising:
polyethyleneoxy and/or polypropyleneoxy groups optionally comprising C₆-C₂₄ alkyl groups, such as the products known as dimethicone copolyol sold by the company Dow Corning under the name DC 1248 or the oils Silwet® L 722, L 7500, L 77 and L 711 by the company Union Carbide, and the (C₁₂)alkylmethicone copolyol sold by the company Dow Corning under the name Q2 5200;
substituted or unsubstituted amine groups, such as the products sold under the name GP 4 Silicone Fluid and GP 7100 by the company Genesee, or the products sold under the names Q2 8220 and Dow Corning 929 or 939 by the company Dow Corning. The substituted amine groups are, in particular, C₁-C₄ aminoalkyl groups;
thiol groups such as the products sold under the names GP 72 A and GP 71 from Genesee;
alkoxylated groups such as the product sold under the name Silicone Copolymer F-755 by SWS Silicones and Abil Wax® 2428, 2434 and 2440 by the company Goldschmidt;
hydroxylated groups such as the polyorgano-siloxanes containing a hydroxyalkyl function, described in French patent application FR-A-85/16334;
acyloxyalkyl groups such as, for example, the polyorganosiloxanes described in patent U.S. Pat. No. 4,957,732.
anionic groups of carboxylic type, such as, for example, in the products described in patent EP 186 507 from the company Chisso Corporation, or of alkylcarboxylic type, such as those present in the product X-22-3701E from the company Shin-Etsu; 2-hydroxyalkylsulfonate; 2-hydroxyalkyl thiosulfate such as the products sold by the company Goldschmidt under the names Abil® S201 and Abil® S255;
hydroxyacylamino groups, such as the polyorgano-siloxanes described in patent application EP 342 834. Mention may be made, for example, of the product Q2-8413 from the company Dow Corning.

The silicones as described above may be used, alone or as a mixture, in an amount of between 0.01% and 20% by weight and preferably between 0.1% and 5% by weight relative to the total weight of the composition.

The composition according to the invention may also comprise ingredients commonly used in cosmetics, such as vitamins, fragrances, nacres, thickeners, polymers other than polymers with a PEG group, gelling agents, trace elements, softeners, sequestrants, fragrances, acidifying or basifying agents, preserving agents, sunscreens, antioxidants, hair-loss counteractants, antidandruff agents, free-radical scavengers and ceramides, or mixtures thereof. Needless to say, a person skilled in the art will take care to select this or these optional additional compound(s), and/or the amount thereof, such that the advantageous properties of the composition according to the invention are not, or are not substantially, adversely affected by the envisioned addition.

Advantageously, the pH of the composition of the present invention is chosen in the range from 2 to 11 and preferentially from 3 to 10, for example from 5 to 8.

The composition according to the invention may comprise a propellant. The propellant is one from among the liquefied or compressed gases usually used for the preparation of aerosol compositions and mixtures thereof. Air, carbon dioxide, compressed nitrogen or a soluble gas such as dimethyl ether, halogenated (in particular fluorinated) hydrocarbons or nonhalogenated hydrocarbons, and mixtures thereof, will preferentially be used.

The composition essentially finds a particularly advantageous application in the haircare field, especially for holding the hair style or shaping the hair. The haircare compositions are preferably shampoos, hair conditioners, hairsetting gels or lotions, blow-drying lotions or fixing and styling compositions such as lacquers or sprays. The compositions may be packaged in various forms, especially in bottles, vaporizers, pump-disperser bottles or aerosol containers in order to allow application of the composition in vaporized form or in the form of a mousse.

In a preferred embodiment, the compositions in accordance with the invention may be used for washing keratin materials such as the hair, the skin, the eyelashes, the eyebrows, the nails, the lips or the scalp, and more particularly the hair.

The compositions according to the invention may be detergent compositions such as shampoos, shower gels and bubble baths. In this embodiment of the invention, the compositions comprise at least 4% by weight relative to the total weight of the composition of at least one anionic and/or nonionic detergent surfactant.

A subject of the invention is thus also a process for treating keratin materials such as the skin or the hair, characterized in that it consists in applying to the keratin materials a cosmetic composition as defined above, and then optionally in rinsing with water.

Thus, this process according to the invention allows hold of the hair style, and care and washing of or makeup-removal from the skin, the hair or any other keratin material.

In another preferred embodiment, the compositions of the invention may be in the form of a rinse-out or leave-in hair conditioner, or alternatively in the form of rinse-out compositions, to be applied before or after any hair treatment, especially dyeing, bleaching, permanent-waving or relaxing of the hair, or alternatively between the two steps of a permanent-waving or hair-relaxing operation.

When the composition is in the form of a hair conditioner optionally to be rinsed out, it advantageously contains at least one cationic surfactant, for example in a concentration generally of between 0.1% and 10% by weight and preferably from 0.5% to 5% by weight relative to the total weight of the composition.

The compositions of the invention may also be in the form of washing compositions for the skin, and in particular in the form of bath or shower solutions or gels, or makeup-removing products.

The compositions according to the invention may also be in the form of aqueous or aqueous-alcoholic lotions for skincare and/or haircare.

The composition according to the invention, after application to human hair and scalp, may be rinsed out or left in after any treatment. It may be in any form conventionally used in the field under consideration, for example in the form of a more or less thickener lotion, a gel, a cream, a spray or a mousse. This composition may be a one-phase or multiphase composition.

According to one preferred embodiment of the invention, the composition may be used as a shampoo.

When the compositions in accordance with the invention are used as standard hair conditioners, they are simply applied to wet hair and the foam generated by massaging or rubbing with the hands is then removed, after an optional action time, by rinsing with water, the operation possibly being repeated one or more times.

The compositions of the invention are illustrated in greater detail in the examples that follow.

### Measurement of the Tg

A film is made using an aqueous solution containing 6% by weight of polymer and dried for 48 hours under a controlled atmosphere at 50% relative humidity and 25° C. The films thus obtained have a thickness of between 10 and 20 μm.

The measuring apparatus is a DSC (TA Instruments).

The sample obtained from the film is placed in a hermetic crucible and is heated according to the following protocol:
equilibrium at initial temperature Ti;
heating 1: raising of the temperature, at a rate of +10° C./min to a final temperature: Tf (° C.);
isotherm for 1 minute;
reducing of the temperature at a rate of −10° C./min to Ti (0° C.);
heating 2: raising of the temperature at a rate of +10° C./min to Tf (0° C.);
isotherm for 1 minute,
   with Ti: initial temperature −120° C.
   with Tf: final temperature +120° C.

The Tg values are measured during the heating steps 1 and 2.

### Measurement of the Water Uptake

About 1 g of dry polymer is placed in an aluminum crucible 4.5 cm in diameter (0.01 m²). It is left to dry for 48 hours in an oven at 60° C. under reduced pressure. The crucibles are removed and weighed immediately (less than one minute after removing from the oven). W1 is obtained.

The crucibles are then placed in a glove box with a given relative humidity (75% HR or 85% HR) and are left therein for 6 hours. They are then weighed again immediately after removing them from the glove box. W2 is obtained.

The water uptake is calculated in the following manner:

[(W2−W1)×100]/W1

### PREPARATION EXAMPLE 1

75 ml of methyl ethyl ketone (MEK) are placed in a reactor (4-necked flask) on which are mounted two addition funnels, a condenser and a mechanical stirrer, and are brought to 80° C.

In parallel, a solution 1 is prepared comprising the monomers: 50 g of polyethylene glycol methacrylate (MPEG 550), 50 g of dimethylaminopropylmethacrylamide (DMAPMA) and the initiator: 0.5 g of (Trigonox 21S).

A solution 2 is also prepared, comprising 75 ml of methyl ethyl ketone and 0.5 g of initiator (Trigonox 21S).

Solution 1 is poured dropwise over 1 hour and solution 2 over two hours, into the 4-necked flask reactor. The resulting mixture is then maintained at 80° C. for 5 hours. The orange-yellow solution obtained is cooled. 95 g of polymer are obtained.

The polymer has a Brookfield viscosity at 15% in MEK, at 25° C., measured with a No. 1 needle-type spindle, at a speed of 0.1 rpm, of 7.5 mPa·s.

The polymer may then be neutralized in the following manner: 290 ml of 1N HCl are added with stirring to the 95 g of polymer and 200 ml of distilled water. The solvent (MEK) is then evaporated off.

The neutralized polymer is soluble in water (at least up to 50% by weight). Its Tg is −60° C.

The neutralized polymer has a water uptake at 85% HR of 51%.

### PREPARATION EXAMPLE 2

100 ml of water are placed in a reactor (4-necked flask) on which are mounted two addition funnels, a condenser and a mechanical stirrer, and are brought to 80° C.

In parallel, a solution 1 comprising 50 g of monomer MPEG 550, 1 g of initiator (potassium persulfate KPS) and 50 ml of water is prepared.

A solution 2 comprising 50 g of monomer DMAPMA 100% neutralized with betaine hydrochloride, and 50 g of water, is also prepared.

Solutions 1 and 2 are poured into the 4-necked flask over 1 hour. After 1 hour at 80° C., a mixture of 1 g of KPS in 50 ml of water is added dropwise thereto over 15 minutes.

The resulting mixture is then maintained at 80° C. for 3 hours. 90 g of polymer neutralized with betaine hydrochloride are obtained.

The polymer has a Brookfield viscosity at 15% in water, at 25° C., measured with a No. 1 needle-type spindle, at a speed of 6 rpm, of 164 mPa·s.

The polymer is soluble in water (at least up to 50% by weight).

Its Tg is −60° C. The neutralized polymer has a water uptake at 85% HR of 90%.

### PREPARATION EXAMPLES 3 TO 17

The following polymers, which are according to the invention or comparative, are prepared according to the process of example 1 (solvent process) or of example 2 (process in water):

| | | Process | |
|---|---|---|---|
| | | and | |
| | | neutrali- | |
| Example | Monomers | zation | Solubility |
| Example 3 | 10% MPEG 550 | Process 1 | Water |
| | 90% DMAPMA | HCl | |
| Example 4 | 25% MPEG 1100 | Process 1 | Water |
| | 75% DMAPMA | HCl | |
| Example 5 | 50% MPEG 1100 | Process 1 | Water |
| | 50% DMAPMA | HCl | |
| Example 6 | 50% MPEG 550 | Process 1 | Water |
| | 50% DMAPMA | HCl | |
| Example 7 | 50% MPEG 550 | Process 2 | Water |
| | 50% SPE | No | |
| | | neutrali- | |
| | | zation | |
| Example 8 | 50% MPEG 550 | Process 1 | Water |
| | 50% DMAEMA | HCl | |
| Example 9 | 50% MPEG 550 | Process 1 | Water |
| | 50% Morpholinoethyl | HCl | |
| | methacrylate | | |
| Example 10 | 50% MPEG 2000 | Process 2 | Water |
| | 50% DMAPMA | No | |
| | | neutrali- | |
| | | zation | |
| Example 11 | 50% MPEG 550 | Process 1 | Water |
| | 50% DMAPMA | Betaine | |
| | | hydro- | |
| | | chloride | |
| Example 12 | 40% MPEG 550 | Process 1 | Water |
| | 50% DMAPMA | HCl | |
| | 10% EEMA | | |
| Example 13 | 40% MPEG 550 | Process 2 | Water |
| | 50% DMAPMA | HCl | |
| | 10% Hydroxyethyl | | |
| | acrylate | | |
| Example 14 | 40% MPEG 550 | Process 1 | Water |
| | 50% DMAPMA | HCl | |
| | 10% Vinylpyrrolidone | | |
| Example 15 | 40% MPEG 550 | Process 1 | Water |
| | 35% DMAPMA | HCl | |
| | 15% Acrylic acid | | |
| Example 16 | 40% MPEG 550 | Process 1 | Water |
| | 10% | HCl | |
| | Tetrahydrofurfuryl | | |
| | methacrylate | | |
| | 50% DMAPMA | | |
| Example 17 | 40% MPEG 550 | Process 1 | Water |
| | 10% Vinylcaprolactam | HCl | |
| | 50% DMAPMA | | |
| Example 18 | 50% MPEG 550 | Process 1 | Dispersible |
| | 50% DMAPMA | 20% | in water |
| | | Behenic | |
| | | acid | |
| Example 19 | 50% MPEG 550 | Process 1 | Dispersible |
| | 50% DMAPMA | Oleic acid | in water |
| Example 20 | 50% MPEG 8000 | Process 1 | Water |
| | 50% DMAPMA | Postneutrali- | |
| | | zation | |
| | | betaine | |
| | | hydrochloride | |
| Example 21 | 25% MPEG 5000 | Process 1 | Water |
| | 75% DMAPMA | Preneutrali- | |
| | | zation | |
| | | betaine | |
| | | hydrochloride | |
| Example 22 | 50% MPEG 4000 | Process 1 | Water |
| | 50% DMAPMA | betaine | |
| | | hydrochloride | |
| Example 23 | 20% MPEG 8000 | Process 1 | Water |
| | 80% DMAPMA | betaine | |
| | | hydrochloride | |

MPEG: polyethylene glycol methacrylate (with MW = 550, 1100 or 2000)
DMAPMA: dimethylaminopropylmethacrylamide
SPE: N,N-dimethyl-N-(2-methacryloyloxyethyl)-N-(3-sulfopropyl)ammonium betaine
DMAEMA: dimethylaminoethyl methacrylate
EEMA: ethoxyethyl methacrylate

### Examples of Comparative Polymers

The following polymers not in accordance with the invention are prepared according to example 1:

| | | |
|---|---|---|
| Comparative 1 | 35% MPEG 550 | Process 1 |
| (hydrophobic | 50% DMAPMA | HCl |
| additional | 15% Ethylhexyl acrylate | |
| monomer) | | |
| Comparative 2 | 50% MPEG 550 | Process 1 |
| (crosslinked | 50% DMAPMA | HCl |
| polymer) | 1% Butanediol dimethacrylate | |
| Comparative 3 | 50% MPEG 550 | |
| (quaternized | 50% TMEACL* | |
| polymer) | | |

*TMEACL: 2-(dimethylamino)ethyl acrylate, quaternized with methyl chloride.

### Examples of Compositions

### Examples of Shampoos According to the Invention

The invention may be illustrated with the nonexhaustive compositions that follow. The compositions described below are not limiting. The percentages are expressed as weight percentages of active material.

| | Ex 20 | Ex 21 | Ex 22 | Ex 23 |
|---|---|---|---|---|
| SODIUM LAURETH | 8% AM | 8% AM | 8% AM | 8% AM |
| SULFATE [1] | | | | |
| COCO-GLUCOSIDE [2] | 5% AM | 5% AM | 5% AM | 5% AM |
| COCOBETAINE [3] | 2% AM | 2% AM | 2% AM | 2% AM |
| POLYQUATERNIUM 10 [6] | 0.5% AM | — | — | 0.5% AM |
| DMAPMA/MPEG 550 | 0.5% AM | 0.5% AM | 0.5% AM | 0.5% AM |
| (50/50) [7] | | | | |
| GUAR | — | 0.1% AM | — | — |
| HYDROXYPROPYLTRI- | | | | |
| MONIUM CHLORIDE [8] | | | | |
| DIMETHICONE [9] | — | 2% AM | — | — |
| DIMETHICONE [10] | — | — | 2% AM | 1.0% AM |
| COCOAMIDE MIPA [13] | — | — | 1.5% AM | 1.5% AM |
| PRESERVING AGENT | Qs | Qs | Qs | Qs |
| FRAGRANCE | Qs | Qs | Qs | Qs |
| SODIUM | Qs pH | Qs pH | Qs pH | Qs pH |
| HYDROXIDE/CITRIC ACID | 6 | 7 | 7 | 7 |
| WATER | Qs | Qs | Qs | Qs |
| | 100% | 100% | 100% | 100% |

| | Ex 24 | Ex 25 | Ex 26 |
|---|---|---|---|
| SODIUM LAURETH SULFATE | 8% AM | 8% AM | 8% AM |
| [1] | | | |
| COCO-GLUCOSIDE [2] | 5% AM | 5% AM | 5% AM |
| COCOBETAINE [3] | 2% AM | 2% AM | 2% AM |
| POLYQUATERNIUM 10 [6] | — | — | — |
| DMAPMA/MPEG 550 | 0.5% AM | 0.5% AM | 0.5% AM |
| (50/50) [7] | | | |
| GUAR | 0.2% AM | 0.2% AM | — |
| HYDROXYPROPYLTRI- | | | |
| MONIUM CHLORIDE [8] | | | |
| DIMETHICONE [11] | 2% AM | — | — |
| AMODIMETHICONE [12] | — | 2% AM | 3% AM |
| COCOAMIDE MIPA [13] | 1.5% AM | 1.5% AM | 1.5% AM |
| PRESERVING AGENT | qs | qs | qs |
| FRAGRANCE | qs | qs | qs |
| SODIUM | qs | qs pH | qs pH |
| HYDROXIDE/CITRIC ACID | pH 7 | 7 | 7 |
| WATER | qs | qs | qs |
| | 100% | 100% | 100% |

[1] Texapon N 702 (Cognis)
[2] Plantacare 818 UP (Cognis)
[3] Dehyton AB 30 (Cognis)
[6] Ucar polymer JR400 LT (Amerchol)
[7] Polymer of example 11
[8] Jaguar C13S (Rhodia)
[9] Belsil DM 300 000 (Wacker)
[10] Mirasil DM 500 000 (Rhodia)
[11] Dow Corning 200 fluid 60 000 (Dow Corning)
[12] Dow Corning 939 emulsion (Dow Corning)
[13] Empilan CIS (Huntsman)

| Constituent | Ex. 27 | Ex. 28 | Ex. 29 | Ex. 30 |
|---|---|---|---|---|
| SODIUM LAURETH SULFATE [1] | 10% AM | 10% AM | 10% AM | 10% AM |
| LAURETH-12 [16] | 4% AM | 4% AM | 4% AM | 4% AM |
| POLYQUATERNIUM 10 [6] | 0.5% AM | — | — | 0.5% AM |
| DMAPMA/MPEG 550 (50/50) [7] | 0.5% AM | 0.5% AM | 0.5% AM | 0.5% AM |
| GUAR HYDROXYPROPYLTRI- | — | 0.1% AM | — | — |
| MONIUM CHLORIDE [8] | | | | |
| DIMETHICONE [9] | — | 2% AM | — | — |
| DIMETHICONE [10] | — | — | 2% AM | 1% AM |
| COCAMIDE MIPA [13] | — | — | 1.5% AM | 1.5% AM |
| PRESERVING AGENT | qs | qs | qs | qs |
| FRAGRANCE | qs | qs | qs | qs |
| SODIUM HYDROXIDE/CITRIC | qs pH 6 | qs pH 7 | qs pH 7 | qs pH 7 |
| ACID | | | | |
| WATER | qsp 100% | qsp 100% | qsp 100% | qsp 100% |

| Constituent | Ex. 31 | Ex. 32 | Ex. 33 |
|---|---|---|---|
| SODIUM LAURETH SULFATE [1] | 10% AM | 10% AM | 10% AM |
| LAURETH-12 [16] | 4% AM | 4% AM | 4% AM |
| DMAPMA/MPEG 550 (50/50) [7] | 0.5% AM | 0.5% AM | 0.5% AM |
| GUAR HYDROXYPROPYLTRI- | 0.2% AM | 0.2% AM | — |
| MONIUM CHLORIDE [8] | | | |
| DIMETHICONE [11] | 2% AM | — | — |
| AMODIMETHICONE [12] | — | 2% AM | 3% AM |
| COCAMIDE MIPA [13] | 1.5% AM | 1.5% AM | 1.5% AM |
| PRESERVING AGENT | qs | qs | qs |
| FRAGRANCE | qs | qs | qs |
| SODIUM HYDROXIDE/CITRIC | qs pH 7 | qs pH 7 | qs pH 7 |
| ACID | | | |
| WATER | qsp 100% | qsp 100% | qsp 100% |

| Constituent | Ex. 34 | Ex. 35 | Ex. 36 | Ex. 37 |
|---|---|---|---|---|
| LAURETH-5 CARBOXYLIC ACID | 6% AM | 6% AM | 6% AM | 6% AM |
| [14] | | | | |
| COCOGLUCOSIDE [2] | 11% AM | 11% AM | 11% AM | 11% AM |
| POLYQUATERNIUM 10 [6] | 0.5% AM | — | — | 0.5% AM |
| DMAPMA/MPEG 550 (50/50) [7] | 0.5% AM | 0.5% AM | 0.5% AM | 0.5% AM |
| GUAR HYDROXYPROPYLTRI- | — | 0.1% AM | — | — |
| MONIUM CHLORIDE [8] | | | | |
| DIMETHICONE [9] | — | 2% AM | — | — |
| DIMETHICONE [10] | — | — | 2% AM | 1% AM |
| COCAMIDE MIPA [13] | — | — | 1.5% AM | 1.5% AM |
| PRESERVING AGENT | qs | qs | qs | qs |
| FRAGRANCE | qs | qs | qs | qs |
| SODIUM HYDROXIDE/CITRIC | qs pH 6 | qs pH 7 | qs pH 7 | qs pH 7 |
| ACID | | | | |
| WATER | qsp 100% | qsp 100% | qsp 100% | qsp 100% |

| Constituent | Ex. 38 | Ex. 39 | Ex. 40 |
|---|---|---|---|
| LAURETH-5 CARBOXYLIC ACID | 6% AM | 6% AM | 6% AM |
| [14] | | | |
| COCOGLUCOSIDE [2] | 11% AM | 11% AM | 11% AM |
| DMAPMA/MPEG 550 (50/50) [7] | 0.5% AM | 0.5% AM | 0.5% AM |
| GUAR HYDROXYPROPYLTRI- | 0.2% AM | 0.2% AM | — |
| MONIUM CHLORIDE [8] | | | |
| DIMETHICONE [11] | 2% AM | — | — |
| AMODIMETHICONE [12] | — | 2% AM | 3% AM |
| COCAMIDE MIPA [13] | 1.5% AM | 1.5% AM | 1.5% AM |
| PRESERVING AGENT | qs | qs | qs |
| FRAGRANCE | qs | qs | qs |
| SODIUM HYDROXIDE/CITRIC | qs pH 7 | qs pH 7 | qs pH 7 |
| ACID | | | |
| WATER | qsp 100% | qsp 100% | qsp 100% |
| Constituent | Ex. 41 | Ex. 42 | Ex. 43 |
| AMMONIUM LAURYL SULFATE | 15% AM | 15% AM | 15% AM |
| [15] | | | |
| COCOGLUCOSIDE [2] | 5% AM | 5% AM | 5% AM |
| POLYQUATERNIUM 10 [6] | — | — | — |
| DMAPMA/MPEG 550 (50/50) [7] | 0.5% AM | 0.5% AM | 0.5% AM |
| GUAR HYDROXYPROPYLTRI- | 0.2% AM | 0.2% AM | — |
| MONIUM CHLORIDE [8] | | | |
| DIMETHICONE [11] | 2% AM | — | — |
| AMODIMETHICONE [12] | — | 2% AM | 3% AM |
| COCAMIDE MIPA [13] | 1.5% AM | 1.5% AM | 1.5% AM |
| PRESERVING AGENT | qs | qs | qs |
| FRAGRANCE | qs | qs | qs |
| SODIUM HYDROXIDE/CITRIC | qs pH 7 | qs pH 7 | qs pH 7 |
| ACID | | | |
| WATER | qsp 100% | qsp 100% | qsp 100% |

[1] Texapon N 702 (Cognis)
[2] Plantacare 818 UP (Cognis)
[3] Dehyton AB 30 (Cognis)
[4] Tego betaine F50 (Goldschmidt)
[5] Miranol C 2M - conc. NP (Rhodia)
[6] Ucar polymer JR400 LT (Amerchol)
[7] Polymer of Preparation Ex. 11
[8] Jaguar C13S (Rhodia)
[9] Belsil DM 300 000 (Wacker)
[10] Mirasil DM 500 000 (Rhodia)
[11] Dow Corning 200 fluid 60 000 (Dow Corning)
[12] Dow Corning 939 emulsion (Dow Corning)
[13] Empilan CIS (Huntsman)
[14] AKYPO RLM 45 CA (Kao)
[15] EMPICOL AL 30/FL (Huntsman)
[16] REWOPAL 12 (Goldschmidt)

The DMAPMA/MPEG 550 (50/50) polymer [7] may be replaced with the polymers of Preparation Examples 1 to 23.

### EXAMPLE 44

A composition was prepared comprising the following constituents (weight %):
7.5% sodium lauryl ether sulfate
2.5% cocobetaine amphoteric surfactant (Dehyton AB30 from Cognis)
5% cocopolyglucoside surfactant (Plantacare 818 UP from Cognis) 11.5% polymer of example 11
qsp 100% water

The shampoo composition obtained provides a good styling effect and the cosmetic properties on dry hair are particularly good.

### EXAMPLE 45

A composition was prepared comprising the following constituents (weight %):
7.5% sodium lauryl ether sulfate
2.5% cocobetaine amphoteric surfactant
5% cocopolyglucoside surfactant
1.5% neutralized polymer of example 18
qsp 100% water

The results are collated in the table below (test performed for the composition of example 45, comprising the polymer of example 18, and for the composition of example 44, comprising the polymer of example 11).

| | Detan- | Smooth- | Detan- | | |
|---|---|---|---|---|---|
| | gling | ness | gling | | |
| | wet hair | wet hair | dry hair | Sheen | Bounce |
| Composition | +++ | +++ | +++ | +++ | ++ |
| according | | | | | |
| to example | | | | | |
| 44 | | | | | |
| Composition | ++++ | ++++ | | ++++ | |
| according | | | | | |
| to example | | | | | |
| 45 | | | | | |
| Control | ++ | ++ | ++ | ++ | 0 |
| (DOP | | | | | |
| camomile | | | | | |
| shampoo | | | | | |

1-50. (canceled)

51. A cosmetic composition, comprising, in a cosmetically acceptable aqueous medium:
I) at least one anionic surfactant and at least one nonionic surfactant, and
II) at least one ethylenic copolymer comprising, as a weight percentage relative to the total weight of the polymer:
a) 10-60% by weight of at least one monomer of formula (I): 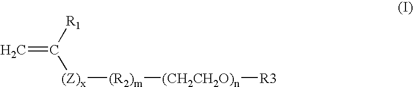
wherein:
R₁ is chosen from a hydrogen atom and a linear or branched hydrocarbon-based radical CₚH₂ₚ₊₁, wherein p is an integer from 1 to 12 inclusive;
Z is a divalent group chosen from —COO—, —CONH—, —CONCH₃—, —OCO—, —O—, —SO₂—, —CO—O—CO—, and —CO—CH₂—CO—;
x is 0 or 1;
R₂ is a saturated or unsaturated, optionally aromatic, linear, branched or cyclic carbon-based divalent radical of 1 to 30 carbon atoms, optionally comprising 1 to 18 heteroatoms chosen from O, N, S, F, Si, and P;
m is 0 or 1;
n is an integer from 3 to 300 inclusive;
R3 is chosen from a hydrogen atom and a saturated or unsaturated, optionally aromatic, linear, branched or cyclic carbon-based radical of 1 to 30 carbon atoms, optionally comprising 1 to 20 heteroatoms chosen from O, N, S, F, Si, and P;
and salts thereof;
b) 40-90% by weight of at least one essentially cationic monomer or salt thereof, chosen from:
(i) at least one cationic monomer of formula (IIa),
(ii) at least one amphoteric monomer of formulae (IIc) and (IId), and
(iii) a mixture of at least one cationic monomer of formula (IIa) with at least one anionic monomer chosen from maleic anhydride, at least one monomer of formula (IIb), and a mixture thereof; and/or
with at least one amphoteric monomer chosen from the amphoteric monomers of formulae (IIc) and (IId); 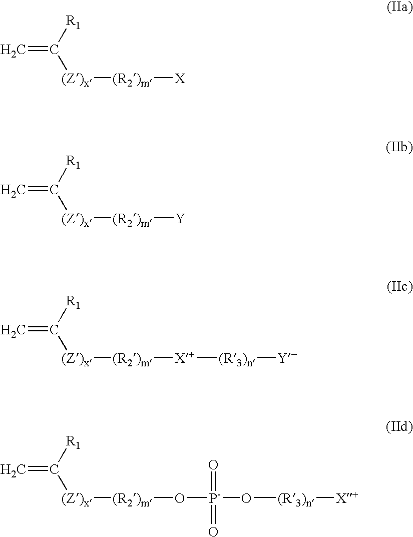
wherein:
R₁ is chosen from a hydrogen atom and a linear or branched hydrocarbon-based radical CₚH₂ₚ₊₁, wherein p is an integer from 1 to 12 inclusive;
Z′ is a divalent group chosen from —COO—, —CONH—, —CONCH₃—, —OCO—, —O—, —SO₂—, —CO—O—CO—, and —CO—CH₂—CO—;
x′ is 0 or 1;
R₂′ is a saturated or unsaturated, optionally aromatic, linear, branched or cyclic divalent carbon-based radical of 1 to 30 carbon atoms, optionally comprising 1 to 18 heteroatoms chosen from O, N, S, F, Si, and P;
m′ is 0 or 1;
in formula (IIa), X is chosen from
(a) a group of formula —N(R₆)(R₇) or —P(R₆)(R₇) or —P⁺R₆R₇R₈, wherein R₆, R₇, and R₈, which may be identical or different, are chosen from
(i) a hydrogen atom; and
(ii) a linear, branched or cyclic, saturated or unsaturated, optionally aromatic alkyl group comprising 1 to 18 carbon atoms, optionally comprising 1 to 10 heteroatoms chosen from O, N, S, F, Si, and P;
or alternatively, (iii) R₆ and R₇ can form, together with the nitrogen or phosphorus atom, a first saturated or unsaturated, optionally aromatic ring comprising in total 5, 6, 7, or 8 atoms; wherein said first ring is optionally fused with at least one other saturated or unsaturated, optionally aromatic ring comprising 5, 6, or 7 atoms; and
(b) a group of formula —R′6-N—R′7-, wherein R′6 and R′7 form, together with the nitrogen atom, a saturated or unsaturated, optionally aromatic ring comprising in total 5, 6, 7, or 8 atoms; wherein said ring is optionally fused with at least one other saturated or unsaturated, optionally aromatic ring comprising 5, 6, or 7 atoms;
Y is a group chosen from —COOH, —SO₃H, —OSO₃H, —PO₃H₂, and —OPO₃H₂;
X′⁺ is a divalent group of formula —N⁺(R₆)(R₇)—, wherein R₆ and R₇, which may be identical or different, are chosen from
(i) a hydrogen atom, and
(ii) a linear, branched or cyclic, optionally aromatic alkyl group, comprising 1 to 25 carbon atoms, optionally comprising 1 to 20 heteroatoms chosen from O, N, S, and P; or
alternatively (iii) R₆ and R₇ can form, together with the nitrogen atom, a first saturated or unsaturated, optionally aromatic ring comprising in total 5, 6, 7, or 8 atoms; said first ring being optionally fused with at least one other saturated or unsaturated, optionally aromatic ring comprising 5, 6, 7, or 8 atoms;
Y′⁻ is a group chosen from —COO⁻, —SO₃⁻, —OSO₃⁻, —PO₃²⁻, and —OPO₃²⁻;
R′3 is a saturated or unsaturated, optionally aromatic, linear, branched or cyclic divalent carbon-based radical of 1 to 30 carbon atoms, optionally comprising 1 to 18 heteroatoms chosen from O, N, S, F, Si, and P;
n′ ranges from 1 to 100; —X″⁺ is a group of formula —N⁺R₆R₇R₈, wherein R₆, R₇, and R₈, which may be identical or different, are chosen from
(i) a hydrogen atom, and
(ii) a linear, branched or cyclic, optionally aromatic alkyl group, comprising 1 to 18 carbon atoms, optionally comprising 1 to 5 heteroatoms chosen from O, N, S, and P; or
alternatively (iii) R₆ and R₇ can form, together with the nitrogen atom, a first saturated or unsaturated, optionally aromatic ring comprising in total 5, 6, or 7 atoms; said first ring being optionally fused with at least one other saturated or unsaturated, optionally aromatic ring comprising 5, 6, or 7 atoms;
c) and optionally 0-50% by weight of at least one nonionic hydrophilic monomer, with the exclusion of methyl acrylate, methyl methacrylate and isopropyl acrylate if they are present in an amount of greater than or equal to 10% by weight.

52. The cosmetic composition of claim 51, wherein R₁ in formula (I) is chosen from a hydrogen atom and a radical chosen from methyl, ethyl, propyl, and butyl radicals.

53. The cosmetic composition of claim 51, wherein Z in formula (I) is chosen from COO and CONH.

54. The cosmetic composition of claim 51, wherein R₂ in formula (I) is chosen from:
an alkylene radical;
an ortho, meta or para-phenylene radical —C₆H₄—, optionally substituted with a C1-C12 alkyl radical optionally comprising 1 to 25 heteroatoms chosen from O, N, S, F, Si, and P; or alternatively a benzylene radical —C₆H₄—CH₂—, optionally substituted with a C1-C12 alkyl radical optionally comprising 1 to 8 heteroatoms chosen from O, N, S, F, Si, and P;
a pyridinium radical of formula: 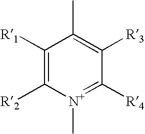
wherein R₁₁ to R₁₄, which may be identical or different, are chosen from a hydrogen atom and a C₁-C₁₂ alkyl radical optionally comprising 1 to 8 heteroatoms chosen from O, N, S, F, Si, and P;
a radical of formula —CH₂—CHOH—, —CH₂—CH₂—CHOH—, CH₂—CH₂—CH(NH₂)—, —CH₂—CH(NH₂)—, —CH₂—CH₂—CH(NHR′)—, —CH₂—CH(NHR′)—, —CH₂—CH₂—CH(NR′R″)—, —CH₂—CH(NR′R″)—, —CH₂—CH₂—CH₂—NR′—, —CH₂—O—CO—O—, CH₂—CH₂—O—CO—O—, —CH₂—CO—O—, —CH₂—CH₂—CO—O—, —CH₂—O—CO—NH—, —CH₂—CH₂—O—CO—NH—, —CH₂—NH—CO—NH—, —CH₂—CH₂—NH—CO—NH—, —CH₂—CH₂—CH₂—O—, and —CH₂—CH₂—CHR′—O—, wherein R′ and R″, which may be identical or different, are each a linear or branched C₁-C₂₂ alkyl radical optionally comprising 1 to 12 heteroatoms chosen from O, N, S, F, Si, and P; and
a mixture of these radicals.

55. The cosmetic composition of claim 51, wherein, for X of formula (IIa),
said first aromatic rings having 5 to 8 total atoms comprise 4 to 6 carbon atoms and/or 2 to 4 heteroatoms chosen from O, S, and N, and
said other aromatic rings having 5 to 7 total atoms comprise 4 to 7 carbon atoms and/or 2 to 4 heteroatoms chosen from O, S, and N.

56. The cosmetic composition of claim 51, wherein, for X′⁺ of formula (IIc),
said first aromatic rings having 5 to 8 total atoms comprise 4 to 6 carbon atoms and/or 2 to 4 heteroatoms chosen from O, S, and N, and
said other aromatic rings having 5 to 8 total atoms comprise 4 to 7 carbon atoms and/or 2 or 3 heteroatoms chosen from O, S, and N.

57. The cosmetic composition of claim 51, wherein, for X″⁺ of formula (IId),
said first aromatic rings having 5 to 7 total atoms comprise 4 to 6 carbon atoms and/or 2 or 3 heteroatoms chosen from O, S, and N, and
said other aromatic rings having 5 to 7 total atoms comprise 4 to 7 carbon atoms and/or 2 or 3 heteroatoms chosen from O, S, and N.

58. The cosmetic composition of claim 51, wherein in formula (I), n ranges from 5 to 200.

59. The cosmetic composition of claim 58, wherein n ranges from 9 to 50.

60. The cosmetic composition of claim 51, wherein in formula (I), R3 is chosen from a hydrogen atom; a succinimido, maleimido, mesityl, tosyl, triethoxysilane, phthalimide, or —CH₂—CH₂CN radical; a benzyl or phenyl radical optionally substituted with a C₁-C₁₂ alkyl radical optionally comprising 1 to 8 heteroatoms chosen from O, N, S, F, Si, and P; a C₁-C₃₀ alkyl radical, optionally comprising 1 to 18 heteroatoms chosen from O, N, S, F, Si, and P;
wherein said benzyl, phenyl or alkyl radicals also optionally comprise a function chosen from succinimido, glutarate-succinimido, glutarate, maleimido, mesityl, benzoate, tosyl, triethoxysilane, phthalimide, thioester, benzotriazole carbonate, butyraldehyde, acetaldehyde diethyl acetal, biotin, phospholipid, succinate, N-hydroxysuccinimide, —SO₃H, —COOH, —PO₄, —NR5R6, and —N⁺R5R6R7, wherein R5, R6, and R7, which may be identical or different, are chosen from a hydrogen atom and a linear, branched or cyclic C₁-C₁₈ alkyl radical, optionally comprising at least one heteroatom or alternatively at least one protecting group.

61. The cosmetic composition of claim 60, wherein said at least one protecting group is chosen from t-butyloxycarbonyl and 9-fluorenylmethoxycarbonyl.

62. The cosmetic composition of claim 51, wherein said at least one monomer of formula (I) is chosen from:
poly(ethylene glycol) (meth)acrylate;
methylpoly(ethylene glycol) (meth)acrylate;
alkylpoly(ethylene glycol) (meth)acrylate;
phenylpoly(ethylene glycol) (meth)acrylate;
the following monomer: 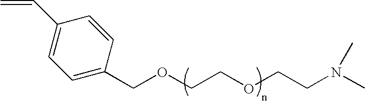
wherein n ranges from 3 to 100; and
a mixture of these monomers.

63. The cosmetic composition of claim 62, wherein n ranges from 7 to 30.

64. The cosmetic composition of claim 51, wherein the at least one monomer of formula (I), alone or as a mixture, is present in an amount of from 20% to 55% by weight relative to the weight of the final polymer.

65. The cosmetic composition of claim 64, wherein the at least one monomer of formula (I), alone or as a mixture is present in an amount of from 30% to 50% by weight relative to the weight of the final polymer.

66. The cosmetic composition of claim 51, wherein, in formulae (IIa), (IIb), (IIc), and/or (IId), the radical R₂′ is chosen from:
an alkylene radical;
a ortho, meta, or para-phenylene radical —C₆H₄—, optionally substituted with at least one C₁-C₁₂ alkyl radical optionally comprising 1 to 25 heteroatoms chosen from N, O, S, F, Si, and/or P;
a benzylene radical —C₆H₄—CH₂—, optionally substituted with at least one C₁-C₁₂ alkyl radical optionally comprising 1 to 25 heteroatoms chosen from O, N, S, F, Si, and P;
a radical chosen from radicals of formula —CH₂—O—CO—O—, CH₂—CH₂—O—CO—O—, —CH₂—CO—O—, —CH₂—CH₂—CO—O—, —[(CH₂)₅—CO—O]ₙ—, —CH₂—CH(CH₃)—O—, —(CH₂)₂—O—, —CH₂—O—CO—NH—, —CH₂—CH₂—O—CO—NH—, —CH₂—NH—CO—NH—, —CH₂—CH₂—NH—CO—NH—, —CH₂—CHOH—, —CH₂—CH₂—CHOH—, —CH₂—CH₂—CH(NH₂)—, —CH₂—CH(NH₂)—, —CH₂—CH₂—CH(NHR′)—, —CH₂—CH(NHR′)—, —CH₂—CH₂—CH(NR′R″)—, —CH₂—CH(NR′R″)—, —CH₂—CH₂—CH₂—NR′—, —CH₂—CH₂—CH₂—O—, and —CH₂—CH₂—CHR′—O—, wherein R′ and R″, which may be identical or different, are each chosen from a linear and branched C₁-C₂₂ alkyl radical optionally comprising 1 to 12 heteroatoms chosen from O, N, S, F, Si, and P; and
a mixture of these radicals.

67. The cosmetic composition of claim 51, wherein, in formula (IIa), the radicals R₆ and R₇ present in X are each independently chosen from a hydrogen atom and a group chosen from methyl, ethyl, propyl, isopropyl, n-butyl, t-butyl, isobutyl, octyl, lauryl, and stearyl groups.

68. The cosmetic composition of claim 51, wherein, in formula (IIa), X is a radical chosen from pyridine, indolyl, isoindolinyl, imidazolyl, imidazolinyl, piperidyl, pyrazolinyl, pyrazolyl, quinoline, pyrazolinyl, pyridyl, piperazinyl, pyrrolidinyl, quinidinyl, thiazolinyl, morpholine, guanidino, amidino and phosphonium radicals, and mixtures thereof.

69. The cosmetic composition of claim 51, wherein the monomers of formula (IIa) are neutralized with at least one neutralizer chosen from neutralizers with a log P of less than or equal to 2 and from agents with a log P of greater than 2.

70. The cosmetic composition of claim 69, wherein the log P of the neutralizers with a log P of less than or equal to 2 ranges from −6 to 0.

71. The cosmetic composition of claim 69, wherein the log P of the agents with a log P of greater than 2 ranges from 3.5 to 10.

72. The cosmetic composition of claim 51, wherein the monomer of formula (IIa) is chosen from:
dimethylaminopropyl(meth)acrylamide, dimethylaminoethyl(meth)acrylamide, diethylaminoethyl (meth)acrylate, dimethylaminoethyl (meth)acrylate, vinylimidazole, vinylpyridine and morpholinoethyl (meth)acrylate, and monomers with the following formulas: 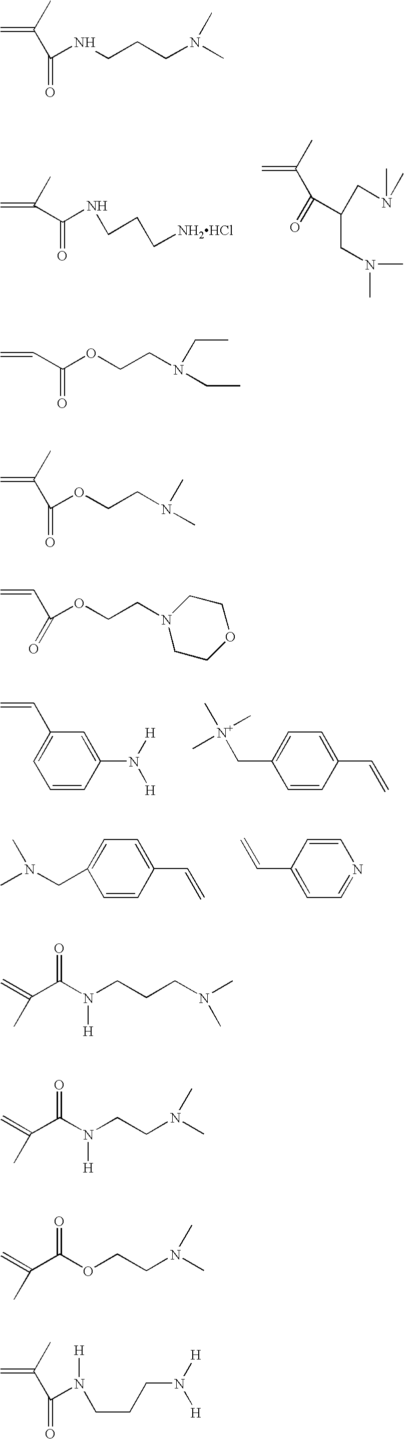 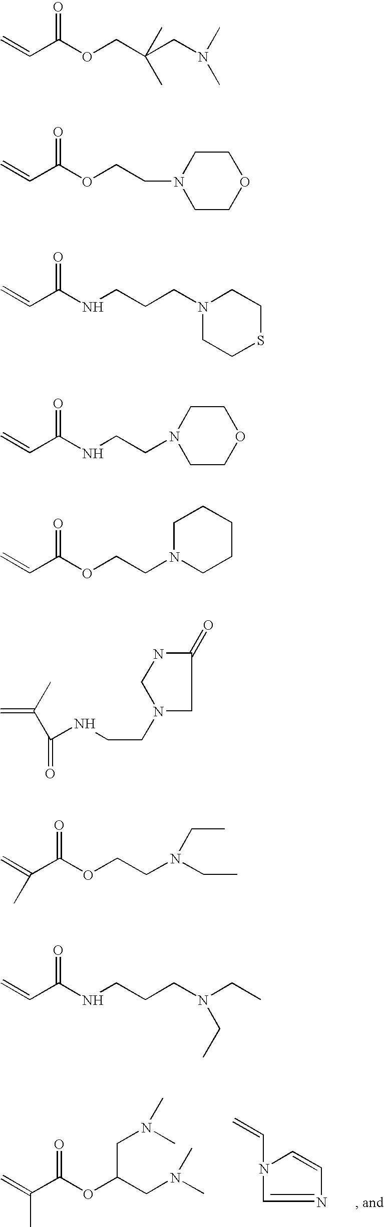
a mixture of these monomers.

73. The cosmetic composition of claim 51, wherein the anionic monomers are chosen from maleic anhydride, acrylic acid, methacrylic acid, crotonic acid, itaconic acid, fumaric acid, maleic acid, 2-carboxyethyl acrylate (CH2=CH—C(O)—O—(CH₂)₂—COOH), styrenesulfonic acid, 2-acrylamido-2-methylpropanesulfonic acid, vinylbenzoic acid, vinylphosphoric acid, and sulfopropyl (meth)acrylate, and salts thereof.

74. The cosmetic composition of claim 51, wherein, in formula (IIc), the radical X′⁺ is chosen from pyridine, indolyl, isoindolinyl, imidazolyl, imidazolinyl, piperidyl, pyrazolinyl, pyrazolyl, quinoline, pyrazolinyl, pyridyl, piperazinyl, pyrrolidinyl, quinidinyl, thiazolinyl, morpholine, guanidino, and amidino radicals, and mixtures thereof.

75. The cosmetic composition of claim 51, wherein, in formulae (IIc) and/or (IId), the radical R13 is chosen from:
an alkylene radical;
a ortho, meta, or para-phenylene radical —C₆H₄—, optionally substituted with at least one C₁-C₁₂ alkyl radical optionally comprising 1 to 5 heteroatoms chosen from O, N, S, F, Si, and P; or alternatively a benzylene radical —C₆H₄—CH₂—, optionally substituted with at least one C₁-C₁₂ alkyl radical optionally comprising 1 to 5 heteroatoms chosen from O, N, S, F, Si, and P;
a radical of formula —CH₂—O—CO—O—, CH₂—CH₂—O—CO—O—, —CH₂—CO—O—, —CH₂—CH₂—CO—O—, —[(CH₂)₅—CO—O]ₙ—, —CH₂—CH(CH₃)—O—, —(CH₂)₂—O—, —CH₂—O—CO—NH—, —CH₂—CH₂—O—CO—NH—, —CH₂—NH—CO—NH—, —CH₂—CH₂—NH—CO—NH—, —CH₂—CHOH—, —CH₂—CH₂—CHOH—, —CH₂—CH₂—CH(NH₂)—, —CH₂—CH(NH₂)—, —CH₂—CH₂—CH(NHR′)—, —CH₂—CH(NHR′)—, —CH₂—CH₂—CH(NR′R″)—, —CH₂—CH(NR′R″)—, —CH₂—CH₂—CH₂—NR′—, —CH₂—CH₂—CH₂—O—; —[CH₂—CH₂O]ₙ—, —[CH₂—CH(CH₃)—O]ₙ—, or —CH₂—CH₂—CHR′—O—, wherein R′ and R″, which may be identical or different, are each independently chosen from a linear and branched C₁-C₂₂ alkyl radical optionally comprising 1 to 12 heteroatoms chosen from O, N, S, F, Si, and P; and
a mixture of these radicals.

76. The cosmetic composition of claim 51, wherein n′ ranges from 1 to 5.

77. The cosmetic composition of claim 51, wherein X″⁺ in formula (IId) is chosen from trimethylammonium, triethylammonium, N,N-dimethyl-N-octylammonium, and N,N-dimethyl-N-laurylammonium radicals.

78. The cosmetic composition of claim 51, wherein the monomers of formula (IIc) and (IId), which may be identical or different, are independently chosen from N,N-dimethyl-N-(2-methacryloyloxyethyl)-N-(3-sulfopropyl)ammonium betaine, N,N-dimethyl-N-(3-methacrylamidopropyl)-N-(3-sulfopropyl)ammonium betaine, 1-(3-sulfopropyl)-2-vinylpyridinium betaine, and 2-methacryloyloxyethylphosphorylcholine.

79. The cosmetic composition of claim 51, wherein, when the essentially cationic monomer is chosen from mixtures of at least one cationic and/or at least one amphoteric monomer with at least one anionic monomer, said at least one anionic monomer being present in an amount ranging from 5% to 40% by weight relative to the weight of the anionic and cationic and/or amphoteric mixture.

80. The cosmetic composition of claim 79, wherein said at least one anionic monomer is present in an amount ranging from 15% to 25% by weight relative to the weight of the anionic and cationic and/or amphoteric mixture.

81. The cosmetic composition of claim 51, wherein the essentially cationic monomer is present in an amount ranging from 45% to 80% by weight relative to the weight of the final polymer.

82. The cosmetic composition of claim 81, wherein the essentially cationic monomer is present in an amount ranging from 50% to 70% by weight relative to the weight of the final polymer.

83. The cosmetic composition of claim 51, wherein the at least one nonionic hydrophilic monomer is present and has a log P ranging from −8 to 2.

84. The cosmetic composition of claim 83, wherein the at least one nonionic hydrophilic monomer is present and has a log P ranging from −6 to 0.

85. The cosmetic composition of claim 51, wherein the at least one nonionic hydrophilic monomer is present and is chosen from those of formula (III), alone or as a mixture: 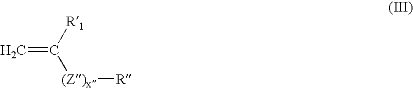
wherein:
R′₁ is chosen from a hydrogen atom and —CH₃;
Z″ is a divalent group chosen from —COO—, —CONH—, —CONCH₃—, —OCO—, —SO₂, —CO—O—CO—, —CO—CH₂—CO—, and —O—;
x″ is 0 or 1;
R″ is a saturated or unsaturated, optionally aromatic, linear, branched or cyclic carbon-based radical of 1 to 30 carbon atoms, optionally comprising 1 to 18 heteroatoms chosen from O, N, S, F, Si, and P.

86. The cosmetic composition of claim 51, wherein the at least one nonionic hydrophilic monomer is present and is chosen from methyl methacrylate, methyl acrylate, ethyl methacrylate, ethyl acrylate, propyl acrylate, isopropyl acrylate, tetrahydrofurfuryl methacrylate, tetrahydrofurfuryl acrylate, 2-hydroxyethyl methacrylate, 2-hydroxyethyl acrylate, ethoxyethyl methacrylate, ethoxyethyl acrylate, N-isopropylacrylamide, N-isopropylmethacrylamide, N,N-dimethylacrylamide, N,N-di-methylmethacrylamide, vinyl acetate, methyl vinyl ether, ethyl vinyl ether, vinylpyrrolidone, vinylcaprolactam, N-vinylacetamide, hydroxylpropyl acrylate, N-vinyl-lactam, acrylamide, N-methylacrylamide, N,N-dimethylacrylamide, N-methyl-N-vinyl-acetamide, N-vinylformamide, N-methyl-N-vinylformamide, and vinyl alcohol, wherein said vinyl alcohol is copolymerized in the form of vinyl acetate and then hydrolyzed.

87. The cosmetic composition of claim 51, wherein the at least one nonionic hydrophilic monomer is present in an amount of from 0.1% to 35% by weight relative to the total weight of the polymer.

88. The cosmetic composition of claim 87, wherein the at least one nonionic hydrophilic monomer is present in an amount of from 5% to 9.5% by weight relative to the total weight of the polymer.

89. The cosmetic composition of claim 51, wherein the copolymer is neutralized.

90. The cosmetic composition of claim 51, wherein the copolymer is neutralized with at least one neutralizer chosen from linear, branched or cyclic aliphatic acids and unsaturated or aromatic acids, containing at least one Brönsted acid function and also optionally comprising at least one heteroatom chosen from O, N, Si, F and P

91. The cosmetic composition of claim 51, wherein said at least one neutralizer comprises from 1 to 1000 carbon atoms.

92. The cosmetic composition of claim 91, wherein said at least one neutralizer comprises from 2 to 500 carbon atoms.

93. The cosmetic composition of claim 51, wherein the copolymer is neutralized with at least one neutralizer chosen from:
at least one linear, branched or cyclic, saturated or unsaturated, optionally aromatic fatty acid comprising 6 to 32 carbon atoms, and comprising at least one COOH or sulfonic acid (—SO₃H) function;
at least one linear, branched or cyclic, saturated or unsaturated, optionally aromatic hydroxy acid, comprising 6 to 32 carbon atoms, and comprising at least one COOH or sulfonic acid (—SO₃H) function;
at least one alkylbenzenesulfonic acid, wherein the alkyl group optionally comprises from 4 to 30 carbon atoms;
at least one amphoteric neutralizer, wherein the alkyl group optionally comprises 4 to 30 carbon atoms; and
a mixture of these radicals.

94. The cosmetic composition of claim 93, wherein the at least one amphoteric neutralizer is chosen from alkylbetaines and alkylamidopropylbetaines.

95. The cosmetic composition of claim 51, wherein the copolymer is neutralized with at least one neutralizer chosen from α-hydroxyethanoic acid, α-hydroxyoctanoic acid, α-hydroxycaprylic acid, ascorbic acid, acetic acid, benzoic acid, behenic acid, capric acid, citric acid, caproic acid, caprylic acid, dodecylbenzenesulfonic acid, 2-ethylcaproic acid, folic acid, fumaric acid, galactaric acid, gluconic acid, glycolic acid, 2-hexadecyleicosanoic acid, hydroxycaproic acid, 12-hydroxystearic acid, isolauric acid (2-butyloctanoic acid), isomyristic acid (2-hexyloctanoic acid), isoarachidic acid (2-octyldodecanoic acid), isolignoceric acid (2-decyltetradecanoic acid), lactic acid, lauric acid, malic acid, myristic acid, oleic acid, palmitic acid, propionic acid, sebacic acid, stearic acid, tartaric acid, terephthalic acid, trimesic acid, undecylenic acid, propylbetaine, cocoamidopropylbetaine, and betaine hydrochloride of formula [(CH₃)₃N+CH₂CO₂H.Cl—], and mixtures thereof.

96. The cosmetic composition of claim 51, wherein the copolymer is neutralized with at least one neutralizer chosen from caproic acid, 2-ethylcaproic acid, oleic acid, behenic acid, stearic acid, acetic acid, citric acid, tartaric acid, betaine hydrochloride, and gluconic acid, and mixtures thereof.

97. The cosmetic composition of claim 90, wherein the at least one neutralizer is added in an amount of from 0.01 to 3 molar equivalents (1% to 300%), relative to the total amine functions of the polymer or of the monomers.

98. The cosmetic composition of claim 97, wherein the at least one neutralizer is added in an amount of from 0.1 to 2 molar equivalents (10% to 200%) relative to the total amine functions of the polymer or of the monomers.

99. The cosmetic composition of claim 97, wherein the at least one neutralizer is added in an amount of from 0.01 to 0.99 molar equivalents (1% to 99%), relative to the total amine functions of the polymer or of the monomers.

100. The cosmetic composition of claim 99, wherein the at least one neutralizer is added in an amount of from 0.1 to 0.8 molar equivalents (10% to 80%), relative to the total amine functions of the polymer or of the monomers.

101. The cosmetic composition of claim 97, wherein the at least one neutralizer is added in an amount of from 0.01 to 3 molar equivalents (1 to 300%), relative to the total amine functions of the polymer or of the monomers.

102. The cosmetic composition of claim 101, wherein the at least one neutralizer is added in an amount of from 0.1 to 2 molar equivalents (10% to 200%), relative to the total amine functions of the polymer or of the monomers.

103. The cosmetic composition of claim 51, wherein the copolymer has a weight-average molecular mass (Mw) ranging from 500 to 5,000,000.

105. The cosmetic composition of claim 104, wherein the copolymer has a weight-average molecular mass (Mw) ranging from 8,000 to 100,000.

106. The cosmetic composition of claim 51, wherein the copolymer comprises:
a monomer of formula (I), alone or as a mixture, present in an amount of from 20% to 80% by weight relative to the weight of the final polymer, and chosen from poly(ethylene glycol) (meth)acrylates; and
an essentially cationic monomer present in an amount of from 40% to 90% by weight relative to the weight of the final polymer, and chosen, alone or as a mixture, from dimethylaminopropyl (meth)acrylamide, dimethylaminoethyl (meth)acrylamide, diethylaminoethyl (meth)acrylate, dimethylaminoethyl (meth)acrylate, vinylimidazole, vinylpyridine, and morpholinoethyl (meth)acrylate; and
the copolymer being neutralized with a neutralizer chosen from 2-ethylcaproic acid, oleic acid, behenic acid, stearic acid, acetic acid, citric acid, tartaric acid, betaine hydrochloride, and gluconic acid, and mixtures thereof.

107. The cosmetic composition of claim 106, wherein the monomer of formula (I), alone or as a mixture, is present in an amount of from 30% to 50% by weight relative to the weight of the final polymer.

108. The cosmetic composition of claim 106, wherein the at least one poly(ethylene glycol) (meth)acrylates have a molecular weight ranging from 350 to 13,000 g/mol.

109. The cosmetic composition of claim 108, wherein the at least one poly(ethylene glycol) (meth)acrylates have a molecular weight ranging from 500 to 8,000 g/mol.

110. The cosmetic composition of claim 106, wherein the essentially cationic monomer is present in an amount of from 50% to 70% by weight relative to the weight of the final polymer.

111. The cosmetic composition of claim 51, wherein the copolymer comprises:
a monomer of formula (I), alone or as a mixture, present in an amount of from 20% to 80% by weight relative to the weight of the final polymer, and chosen from poly(ethylene glycol) (meth)acrylates; and
an essentially cationic monomer present in an amount of from 40% to 90% by weight relative to the weight of the final polymer, and chosen, alone or as a mixture, from at least one dimethylaminopropyl (meth)acrylamide, and
the copolymer being neutralized with at least one neutralizer chosen from behenic acid and betaine hydrochloride.

112. The cosmetic composition of claim 111, wherein the monomer of formula (I), alone or as a mixture, is present in an amount of from 30% to 50% by weight relative to the weight of the final polymer.

113. The cosmetic composition of claim 111, wherein the poly(ethylene glycol) (meth)acrylates have a molecular weight ranging from 350 to 13,000 g/mol.

114. The cosmetic composition of claim 113, wherein the poly(ethylene glycol) (meth)acrylates have a molecular weight ranging from 500 to 8,000 g/mol.

115. The cosmetic composition of claim 111, wherein the essentially cationic monomer is present in an amount of from 50% to 70% by weight relative to the weight of the final polymer.

116. The cosmetic composition of claim 51, wherein the at least one ethylenic copolymer is present in an amount of from 0.01% to 30% by weight of solids relative to the total weight of the composition.

117. The cosmetic composition of claim 116, wherein the at least one ethylenic copolymer is present in an amount of from 0.5% to 3% by weight relative to the total weight of the composition.

118. The cosmetic composition of claim 51, wherein the at least one anionic surfactant is chosen from the salts of alkyl sulfates, alkyl ether sulfates, and alkyl ether carboxylates, and mixtures thereof.

119. The cosmetic composition of claim 51, wherein the at least one anionic surfactant is present in an amount of from 3% to 40% by weight relative to the total weight of the composition.

120. The cosmetic composition of claim 119, wherein the at least one anionic surfactant is present in an amount of from 5% to 25% by weight relative to the total weight of the composition.

121. The cosmetic composition of claim 51, wherein the at least one nonionic surfactant is chosen from polyethoxylated, polypropoxylated, or polyglycerolated fatty acids, alkylphenols, α-diols, and alcohols comprising a fatty chain, and at least one from copolymers of ethylene oxide and of propylene oxide, condensates of ethylene oxide and of propylene oxide with fatty alcohols; polyethoxylated fatty amides and polyglycerolated fatty amides comprising 1 to 5 glycerol groups, and oxyethylenated fatty acid esters of sorbitan comprising 2 to 30 mol of ethylene oxide; fatty acid esters of sucrose, fatty acid esters of polyethylene glycol, alkylpolyglycosides, N-alkylglucamine derivatives, and amine oxides.

122. The cosmetic composition of claim 121, wherein the at least one alcohol comprising a fatty chain comprises 8 to 22 carbon atoms.

123. The cosmetic composition of claim 121, wherein the at least one alcohol comprising a fatty chain comprises from 2 to 50 ethylene oxide or propylene oxide groups and from 2 to 30 glycerol groups.

124. The cosmetic composition of claim 121, wherein the polyethoxylated fatty amides comprise 2 to 30 mol of ethylene oxide.

125. The cosmetic composition of claim 121, wherein the polyglycerolated fatty amides comprise 1.5 to 4 glycerol groups.

126. The cosmetic composition of claim 51, wherein the at least one nonionic surfactant is present in an amount of from 0.5% to 25% by weight relative to the total weight of the cosmetic composition.

127. The cosmetic composition of claim 126, wherein the at least one nonionic surfactant is present in an amount of from 2% to 10% by weight relative to the total weight of the cosmetic composition.

128. The cosmetic composition of claim 51, further comprising at least one amphoteric surfactant.

129. The cosmetic composition of claim 51, wherein the total amount of surfactant ranges from 3.5% to 50% by weight relative to the total weight of the composition.

130. The cosmetic composition of claim 129, wherein the total amount of surfactant ranges from 8% to 25% by weight relative to the total weight of the composition.

131. The cosmetic composition of claim 51, wherein the cosmetically acceptable medium comprises at least one constituent chosen from water; hydrophilic organic solvents; and glycol ethers.

132. The cosmetic composition of claim 131, wherein said hydrophilic organic solvents are chosen from alcohols.

133. The cosmetic composition of claim 132, wherein said alcohols are chosen from linear and branched C₁-C₆ monoalcohols and polyols.

134. The cosmetic composition of claim 51, further comprising at least one cationic polymer or at least one amphoteric polymer.

135. The cosmetic composition of claim 134, wherein the at least one cationic polymer or at least one amphoteric polymer is present in an amount of from 0.001% to 20% by weight relative to the total weight of the final composition.

136. The cosmetic composition of claim 135, wherein the at least one cationic polymer or at least one amphoteric polymer is present in an amount of from 0.01% to 10% by weight relative to the total weight of the final composition.

137. The cosmetic composition of claim 51, further comprising at least one silicone.

138. The cosmetic composition of claim 51, further comprising at least one cationic polymer or at least one amphoteric polymer, and at least one silicone.

139. The cosmetic composition of claim 138, wherein the at least one cationic polymer or at least one amphoteric polymer is present in an amount of from 0.001% to 20% by weight relative to the total weight of the final composition.

140. The cosmetic composition of claim 139, wherein the at least one cationic or at least one amphoteric polymer is present in an amount of from 0.1% to 5% by weight relative to the total weight of the final composition.

141. The cosmetic composition of claim 51, further comprising at least one additive chosen from waxes, pasty fatty substances, gums and mixtures thereof, oils of animal, plant, mineral or synthetic origin, synthetic esters and synthetic ethers; fatty alcohols containing from 12 to 26 carbon atoms; volatile or nonvolatile, linear or cyclic silicone-based oils, which are liquid or pasty at room temperature; polymers other than polymers with a PEG group; vitamins, fragrances, nacres, thickeners, gelling agents, trace elements, softeners, sequestrants, fragrances, acidifying or basifying agents, preserving agents, sunscreens, antioxidants, hair-loss counteractants, antidandruff agents, propellants and ceramides, and mixtures thereof.

142. The cosmetic composition of claim 51, wherein said composition is in the form of a haircare composition for holding the hair style or shaping the hair.

143. The cosmetic composition of claim 142, where said haircare composition is in the form of a shampoo.

144. A process for treating keratin materials, comprising applying to the keratin materials a cosmetic composition, leaving the cosmetic composition on the keratin materials for an optional action time, and then optionally rinsing the keratin materials,
wherein said cosmetic composition comprises, in a cosmetically acceptable aqueous medium:
I) at least one anionic surfactant and at least one nonionic surfactant, and
II) at least one ethylenic copolymer comprising, as a weight percentage relative to the total weight of the polymer:
a) 10-60% by weight of at least one monomer of formula (I): 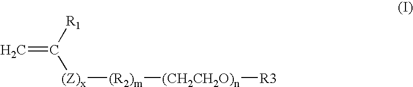
wherein:
R₁ is chosen from a hydrogen atom and a linear or branched hydrocarbon-based radical CₚH₂ₚ₊₁, wherein p is an integer from 1 to 12 inclusive;
Z is a divalent group chosen from —COO—, —CONH—, —CONCH₃—, —OCO—, —O—, —SO₂—, —CO—O—CO—, and —CO—CH₂—CO—;
x is 0 or 1;
R₂ is a saturated or unsaturated, optionally aromatic, linear, branched or cyclic carbon-based divalent radical of 1 to 30 carbon atoms, optionally comprising 1 to 18 heteroatoms chosen from O, N, S, F, Si, and P;
m is 0 or 1;
n is an integer from 3 to 300 inclusive;
R3 is chosen from a hydrogen atom and a saturated or unsaturated, optionally aromatic, linear, branched or cyclic carbon-based radical of 1 to 30 carbon atoms, optionally comprising 1 to 20 heteroatoms chosen from O, N, S, F, Si, and P;
and salts thereof;
b) 40-90% by weight of at least one essentially cationic monomer or salt thereof, chosen from:
(i) at least one cationic monomer of formula (IIa),
(ii) at least one amphoteric monomer of formulae (IIc) and (IId), and
(iii) a mixture of at least one cationic monomer of formula (IIa) with at least one anionic monomer chosen from maleic anhydride, at least one monomer of formula (IIb), and a mixture thereof; and/or
with at least one amphoteric monomer chosen from the amphoteric monomers of formulae (IIc) and (IId); 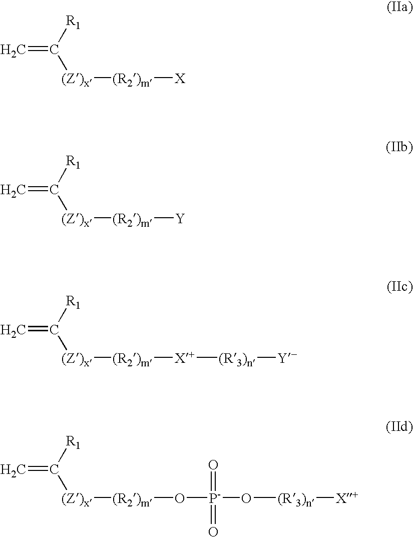
wherein:
R₁ is chosen from a hydrogen atom and a linear or branched hydrocarbon-based radical CₚH₂ₚ₊₁, wherein p is an integer from 1 to 12 inclusive;
Z′ is a divalent group chosen from —COO—, —CONH—, —CONCH₃—, —OCO—, —O—, —SO₂—, —CO—O—CO—, and —CO—CH₂—CO—;
x′ is 0 or 1;
R₂′ is a saturated or unsaturated, optionally aromatic, linear, branched or cyclic divalent carbon-based radical of 1 to 30 carbon atoms, optionally comprising 1 to 18 heteroatoms chosen from O, N, S, F, Si, and P;
m′ is 0 or 1;
in formula (IIa), X is chosen from
(a) a group of formula —N(R₆)(R₇) or —P(R₆)(R₇) or —P+R₆R₇R₈, wherein R₆, R₇, and R₈, which may be identical or different, are chosen from
(i) a hydrogen atom; and
(ii) a linear, branched or cyclic, saturated or unsaturated, optionally aromatic alkyl group comprising 1 to 18 carbon atoms, optionally comprising 1 to 10 heteroatoms chosen from O, N, S, F, Si, and P; or
alternatively (iii) R₆ and R₇ can form, together with the nitrogen or phosphorus atom, a first saturated or unsaturated, optionally aromatic ring comprising in total 5, 6, 7, or 8 atoms; wherein said first ring is optionally fused with at least one other saturated or unsaturated, optionally aromatic ring comprising 5, 6, or 7 atoms; and
(b) a group of formula —R′6-N—R′7-, wherein R′6 and R′7 form, together with the nitrogen atom, a saturated or unsaturated, optionally aromatic ring comprising in total 5, 6, 7, or 8 atoms; wherein said ring is optionally fused with at least one other saturated or unsaturated, optionally aromatic ring comprising 5, 6, or 7 atoms;
Y is a group chosen from —COOH, —SO₃H, —OSO₃H, —PO₃H₂, and —OPO₃H₂;
X′⁺ is a divalent group of formula —N⁺(R₆)(R₇)—, wherein R₆ and R₇, which may be identical or different, are chosen from
(i) a hydrogen atom, and
(ii) a linear, branched or cyclic, optionally aromatic alkyl group, comprising 1 to 25 carbon atoms, optionally comprising 1 to 20 heteroatoms chosen from O, N, S, and P; or
alternatively (iii) R₆ and R₇ can form, together with the nitrogen atom, a first saturated or unsaturated, optionally aromatic ring comprising in total 5, 6, 7, or 8 atoms; said first ring being optionally fused with at least one other saturated or unsaturated, optionally aromatic ring comprising 5, 6, 7, or 8 atoms;
Y′⁻ is a group chosen from —COO⁻, —SO₃⁻, —OSO₃⁻, PO₃²⁻, and OPO₃²⁻;
R′3 is a saturated or unsaturated, optionally aromatic, linear, branched or cyclic divalent carbon-based radical of 1 to 30 carbon atoms, optionally comprising 1 to 18 heteroatoms chosen from O, N, S, F, Si, and P;
n′ ranges from 1 to 100; —X″⁺ is a group of formula —N⁺R₆R₇R₈, wherein R₆, R₇, and R₈, which may be identical or different, are chosen from
(i) a hydrogen atom, and
(ii) a linear, branched or cyclic, optionally aromatic alkyl group, comprising 1 to 18 carbon atoms, optionally comprising 1 to 5 heteroatoms chosen from O, N, S and P; or
alternatively (iii) R₆ and R₇ can form, together with the nitrogen atom, a first saturated or unsaturated, optionally aromatic ring comprising in total 5, 6, or 7 atoms; said first ring being optionally fused with at least one other saturated or unsaturated, optionally aromatic ring comprising 5, 6, or 7 atoms;
c) and optionally 0-50% by weight of at least one nonionic hydrophilic monomer, with the exclusion of methyl acrylate, methyl methacrylate and isopropyl acrylate if they are present in an amount of greater than or equal to 10% by weight.

145. A process according to claim 144, wherein said keratin materials are chosen from bodily or facial skin, nails, bodily hair, head hair, and eyelashes.
